SEQUENCE LISTING (3 sequences)

SEQ ID NO: 1 - protein, 581 aa, synthetic construct
MVDCNYSVACNVMVPMRDGVRLAVDLYRPDADGPVPVLLVRNPYDKFDVFAWSTQSTNWLEFVRDGYAVVIQDTRGLFASEGEFVPHVDDECDAEDTLSWILEQAWCDGNVGMFGVSYLGVTQWQAAVSGVGGLKAIAPSMASADLYRAPWYGPGGALSVEALLGWSALIGRQLITSRCDARPEDAADFVQLAAILNDVAGAASVTPLAEQPLLGRLIPWVIDQVVDHPDNDESWQSISLFERLGGLATPALITAGWYDGFVGESLRTFVAVKDNADARLVVGPWSHTNLTGRNADRKFGIAATYPIQEATTMHKAFFDRHLRGETDALAGVPKVRLFVMGIDEWRDETDWPLPDTAYTPFYLGGSGAANTSTGGGTLSTSISGTESADTYLYDPADPVPSLGGTLLFHNGDNGPADQRPIHDRDDVLCYSTEVLTDPVEVTGTVSARLFVSSSAVDTDFTAKLVDVFPDGRAIALCDGIVRMRYRETLVNPTLIEAGEIYEVAIDMLATSNVFLPGHRIMVQVSSSNFPKYDRNSNTGGVIAREQLEEMCTAVNRIHRGPEHPSHIVLPIIKRKLAAALE

SEQ ID NO: 2 - protein, 587 aa, Rhodococcus sp.
MVDGNYSVASNVMVPMRDGVRLAVDLYRPDADGPVPVLLVRNPYDKFDVFAWSTQSTNWLEFVRDGYAVVIQDTRGLFASEGEFVPHVDDEADAEDTLSWILEQAWCDGNVGMFGVSYLGVTQWQAAVSGVGGLKAIAPSMASADLYRAPWYGPGGALSVEALLGWSALIGTGLITSRSDARPEDAADFVQLAAILNDVAGAASVTPLAEQPLLGRLIPWVIDQVVDHPDNDESWQSISLFERLGGLATPALITAGWYDGFVGESLRTFVAVKDNADARLVVGPWSHSNLTGRNADRKFGIAATYPIQEATTMHKAFFDRHLRGETDALAGVPKVRLFVMGIDEWRDETDWPLPDTAYTPFYLGGSGAANTSTGGGTLSTSISGTESADTYLYDPADPVPSLGGTLLFHNGDNGPADQRPIHDRDDVLCYSTEVLTDPVEVTGTVSARLFVSSSAVDTDFTAKLVDVFPDGRAIALCDGIVRMRYRETLVNPTLIEAGEIYEVAIDMLATSNVFLPGHRIMVQVSSSNFPKYDRNSNTGGVIAREQLEEMCTAVNRIHRGPEHPSHIVLPIIKRKLAAALEHHHHHH

SEQ ID NO: 3 - protein, 587 aa, Rhodococcus sp.
MVDGNYSVASNVMVPMRDGVRLAVDLYRPDADGPVPVLLVRNPYDKFDVFAWSTQSTNWLEFVRDGYAVVIQDTRGLFASEGEFVPHVDDEADAEDTLSWILEQAWCDGNVGMFGVSYLGVTQWQAAVSGVGGLKAIAPSMASADLYRAPWYGPGGALSVEALLGWSALIGRQLITSRSDARPEDAADFVQLAAILNDVAGAASVTPLAEQPLLGRLIPWVIDQVVDHPDNDESWQSISLFERLGGLATPALITAGWYDGFVGESLRTFVAVKDNADARLVVGPWSHSNLTGRNADRKFGIAATYPIQEATTMHKAFFDRHLRGETDALAGVPKVRLFVMGIDEWRDETDWPLPDTAYTPFYLGGSGAANTSTGGGTLSTSISGTESADTYLYDPADPVPSLGGTLLFHNGDNGPADQRPIHDRDDVLCYSTEVLTDPVEVTGTVSARLFVSSSAVDTDFTAKLVDVFPDGRAIALCDGIVRMRYRETLVNPTLIEAGEIYEVAIDMLATSNVFLPGHRIMVQVSSSNFPKYDRNSNTGGVIAREQLEEMCTAVNRIHRGPEHPSHIVLPIIKRKLAAALEHHHHHH

(19) United States
(12) Patent Application Publication
(10) Pub. No.: US 2025/0114653 A1
(43) Pub. Date: Apr. 10, 2025

(54) COMPOSITIONS, METHODS, AND ARTICLES COMPRISING COCAINE ESTERASE FOR DETOXIFYING AN ORGANOPHOSPHATE-BASED AGENT

(71) Applicant: Four Winds Biotherapeutics, Inc., San Diego, CA (US)

(72) Inventors: Nicolas VILLANUEVA, San Diego, CA (US); Alexis VASQUEZ, San Diego, CA (US)

(21) Appl. No.: 18/689,050

(22) PCT Filed: Jul. 22, 2022
(86) PCT No.: PCT/US2022/038073

§ 371 Date: Mar. 4, 2024

Related U.S. Application Data
(60) Provisional application No. 63/225,337, filed on Jul. 23, 2021.

(51) Int. Cl. A62D 3/02; A61K 38/46; A62D 3/30; A62D 101/02; A62D 101/04; A62D 101/26; C12N 9/18

(52) U.S. Cl. CPC A62D 3/02; A61K 38/465; A62D 3/30; C12N 9/18; C12Y 301/01084; A62D 2101/02; A62D 2101/04; A62D 2101/26; A62D 2203/02

(57) ABSTRACT

Disclosed herein are compositions, methods, and articles of personal protective equipment for use in detoxifying an organophosphate-based agent, wherein the compositions, methods, and articles comprise contacting the organophosphate-based agent with a cocaine esterase, wherein the contacting detoxifies the organophosphate-based agent.

### CROSS-REFERENCE

This application claims the benefit of U.S. Provisional Patent Application No. 63/225,337, filed Jul. 23, 2021, which is incorporated herein by reference in its entirety.

### BACKGROUND

This application relates to compositions, methods, and articles of personal protective equipment (PPE) comprising a therapeutically-effective amount of a cocaine esterase. The compositions, methods, and PPE disclosed herein are useful for detoxifying an organophosphate-based agent.

### SUMMARY OF INVENTION

Disclosed herein are methods for detoxifying an organophosphate-based agent, wherein the methods comprise: contacting the organophosphate-based agent with a cocaine esterase, wherein the contacting detoxifies the organophosphate-based agent. In some embodiments, the cocaine esterase comprises an amino acid sequence with at least two mutations in SEQ ID NO:1. In some embodiments, the at least two mutations comprise T172R and G173Q. In some embodiments, the cocaine esterase comprises a catalytic triad of aspartate, histidine, and serine. In some embodiments, the cocaine esterase comprises a catalytic triad of glutamate, histidine, and serine. In some embodiments, the methods further comprise adding an oxime compound after contacting the organophosphate-based agent with the cocaine esterase. In some embodiments, the oxime compound assists with catalysis of a cocaine esterase-mediated hydrolysis of the organophosphate-based agent. In some embodiments, the oxime compound is selected from the group consisting of pralidoxime (2-PAM), asoxime (HI-6), deazapralidoxime (DZP), methoxime (MMB4), obidoxime, trimedoxime (TMB4), TAB2OH, ortho-7, and 3-hyroxy-2-pyridinealdoxime. In some embodiments, the organophosphate-based agent comprises a chemical weapon. In some embodiments, the chemical weapon is selected from the group consisting of G-series nerve agents, V-series nerve agents, GV-series nerve agents, carbamates, and fourth generation agents. In some embodiments, the chemical weapon is selected from the group consisting of tabun (GA), sarin (GB), butylsarin, diethyltabun, soman (GD), cyclosarin (GF), Novichok agents A232 and A234, GV, VE, VG, VM, VP, VS, venomous agent X (VX), Chinese VX, Russian VX (VR), EA-3148, EA-2192, 2-dimethylaminoethyl-(dimethylamido)-fluorophosphate), aldicarb, methomyl, EA-3990, EA-4056, substance-33, A230, Novichok-5, Novichok-7, paraoxon, paraoxon-ethyl, paraoxon-methyl, methamidophos, and fenamiphos. In some embodiments, the organophosphate-based agent comprises an organophosphate-based pesticide. In some embodiments, the organophosphate-based agent comprises an organophosphate-based pesticide selected from the group consisting of azamethiphos, azinphos methyl, bomyl, carbamates (aldicarb, methomyl, EA-3990, and EA-4056), carbophenothion, chlorethoxyphos, chlorfenvinphos, chlormephos, chlorpyrifos, chlorpyrifos methyl, chlorthiophos, coumaphos, cyanofenphos, demeton, dialifor, dialkylphosphates (DAPs), diazinon, dichlorvos, dicrotophos, diethyldithiophosphate (DEDTP), diethylphosphate (DEP), dimefos, dimefox, dimethoate, dimethyldithiophosphate (DMDTP), dimethylthiophosphate (DMTP), dioxathion, disulfoton, endothion, EPN, ethion, ethyl parathion, famphur, fenamiphos, fenophosphon, fensulfothion, fenthion, fenitrothion, fonofos, fosthietan, isofenphos, 2-isopropyl-4-methyl-6-hydroxypyrimidine (IMPY), isazophos methyl, malathion, mephosfolan methamidophos, methidathion, methyl parathion, mevinphos, mipafox, monocrotophos, oxydemeton methyl, parathion (or ethyl parathion), paraoxon, phorate, phosfolan, phosmet (imidan), phosphamidon, pirimiphos methyl, prothoate, schradan, sulfotepp, temephos, terbuos, tetrachlorvinphos, tetraethyl pyrophosphate, dimethyl 1,2-dibromo-2,2-dichloroethylphosphate (naled or dibrom), and 3,5,6-trichloro-2-pyridinol (TCPy).

Disclosed herein are also compositions, wherein the compositions comprise a therapeutically effective amount of a cocaine esterase to detoxify an organophosphate-based agent. In some embodiments, the cocaine esterase catalyzes the breakdown of cocaine into metabolite ecgonine methyl ester and benzoic acid. In some embodiments, the cocaine esterase comprises an amino acid sequence with at least two mutations in SEQ ID NO:1. In some embodiments, the at least two mutations comprise T172R and G173Q. In some embodiments, the cocaine esterase comprises a catalytic triad of aspartate, histidine, and serine. In some embodiments, the cocaine esterase comprises a catalytic triad of glutamate, histidine, and serine. In some embodiments, the cocaine esterase is further PEGylated. In some embodiments, the cocaine esterase is active for greater than or equal to 6 h at about 37° C. In some embodiments, maximum initial velocity of a reaction (Vₘₐₓ) of the cocaine esterase ranges from 1200 μmol/min to 12,000 μmol/min. In some embodiments, the compositions further comprise an oxime compound. In some embodiments, the oxime compound is selected from the group consisting of pralidoxime (2-PAM), asoxime (HI-6), deazapralidoxime (DZP), methoxime (MMB4), obidoxime, trimedoxime (TMB4), TAB2OH, ortho-7, and 3-hyroxy-2-pyridinealdoxime. In some embodiments, the compositions further comprise a pharmaceutically acceptable excipient. In some embodiments, the pharmaceutically acceptable excipient is saline. In some embodiments, the composition comprises cocaine esterase in a range from 100 mg to 200 mg.

Disclosed herein are also articles of personal protective equipment (PPE) comprising a cocaine esterase. In some embodiments, the PPE protects a wearer from exposure to nerve agents, chemical weapons, organophosphate pesticides, or a combination thereof. In some embodiments, the PPE is selected from the group consisting of a mask, a helmet, a hat, a cap, a guard, gloves, a footwear, a footwear cover, a jacket, a gown, pants, and a suit. In some embodiments, the PPE comprises a fabric, a plastic, a rubber, a metal, or a combination thereof. In some embodiments, the cocaine esterase is incorporated into the PPE during a manufacturing step.

Disclosed herein are also detoxifying compositions comprising, by weight: 80-99% of a cocaine esterase; and 1-20% of an oxime compound. In some embodiments, the cocaine esterase catalyzes the breakdown of cocaine into metabolite ecgonine methyl ester and benzoic acid. In some embodiments, the cocaine esterase comprises an amino acid sequence with at least two mutations in SEQ ID NO:1. In some embodiments, the at least two mutations comprise T172R and G173Q. In some embodiments, the cocaine esterase comprises a catalytic triad of aspartate, histidine, and serine. In some embodiments, the cocaine esterase comprises a catalytic triad of glutamate, histidine, and serine. In some embodiments, the cocaine esterase is further PEGylated. In some embodiments, the cocaine esterase is active for greater than or equal to 6 h at about 37° C. In some embodiments, maximum initial velocity of a reaction (Vₘₐₓ) of the cocaine esterase ranges from 1200 μmol/min to 12,000 μmol/min. In some embodiments, the oxime compounds comprise pralidoxime, deazapralidoxime, methoxime, obidoxime, trimedoxime, ortho-7, 3-hyroxy-2-pyridinealdoxime, or a combination thereof. In some embodiments, the detoxifying compositions further comprise a pharmaceutically acceptable excipient. In some embodiments, the pharmaceutically acceptable excipient is saline.

Disclosed herein are also methods for manufacturing a cocaine esterase composition, wherein the method comprises: a) obtaining a strain of bacteria producing a recombinant cocaine esterase; b) fermenting the bacteria in a large-scale volume; c) purifying the recombinant cocaine esterase from the large-scale volume; and d) formulating the recombinant cocaine esterase into a cocaine esterase composition, wherein the cocaine esterase composition further comprises an oxime compound. In some embodiments, the strain of bacteria producing a recombinant cocaine esterase comprises E. coli or endotoxin-free E. coli. In some embodiments, time for fermentation ranges from 4 hours to 96 hours. In some embodiments, the large-scale volume ranges from 1 liter to 90,000 liters. In some embodiments, temperature for fermentation ranges from 6° C. to 37° C. In some embodiments, purifying the recombinant cocaine esterase from the large-scale volume comprises extraction, centrifugation, immobilized metal chromatography, ion exchange chromatography, size exclusion chromatography, or a combination thereof. In some embodiments, a purified recombinant cocaine esterase yield after purifying the recombinant cocaine esterase from the large-scale volume ranges from 70% to 95%. In some embodiments, the oxime compound is selected from the group consisting of pralidoxime, deazapralidoxime, methoxime, obidoxime, trimedoxime, and ortho-7, 3-hyroxy-2-pyridinealdoxime. In some embodiments, after step c), the methods further comprise formulating a pharmaceutically acceptable excipient into the cocaine esterase composition. In some embodiments, the pharmaceutically acceptable excipient is saline. In some embodiments, a concentration of the cocaine esterase in the pharmaceutically acceptable excipient ranges from about 1 mg/ml to about 40 mg/ml. In some embodiments, the cocaine esterase catalyzes the breakdown of cocaine into metabolite ecgonine methyl ester and benzoic acid. In some embodiments, the cocaine esterase comprises an amino acid sequence with at least two mutations in SEQ ID NO:3. In some embodiments, the at least two mutations comprise T172R and G173Q. In some embodiments, the cocaine esterase comprises a catalytic triad of aspartate, histidine, and serine. In some embodiments, the cocaine esterase comprises a catalytic triad of glutamate, histidine, and serine. In some embodiments, the cocaine esterase is further PEGylated. In some embodiments, the cocaine esterase is active for greater than or equal to 6 h at 37° C. In some embodiments, maximum initial velocity of a reaction (Vₘₐₓ) of the cocaine esterase ranges from 1200 μmol/min to 12,000 μmol/min.

In some embodiments, in the compositions disclosed herein, upon contacting the cocaine esterase with the organophosphate-based agent, an amount of the organophosphate-based agent is decreased to no more than about 25% of the original amount after no more than about 30 minutes. In some embodiments, the amount of the organophosphate-based agent is measured by liquid chromatography with tandem mass spectrometry (LC-MS/MS).

In some embodiments, in the methods disclosed herein, upon contacting the cocaine esterase with the organophosphate-based agent, an amount of the organophosphate-based agent is decreased to no more than about 25% of the original amount after no more than about 30 minutes. In some embodiments, the amount of the organophosphate-based agent is measured by liquid chromatography with tandem mass spectrometry (LC-MS/MS).

### INCORPORATION BY REFERENCE

All publications, patents, and patent applications mentioned in this specification are herein incorporated by reference in their entirety to the same extent as if each individual publication, patent, or patent application was specifically and individually indicated to be incorporated by reference in their entirety. In the event of a conflict between a term herein and a term in an incorporated reference, the term herein controls.

### BRIEF DESCRIPTION OF THE DRAWINGS

The novel features of the compositions, methods, and articles disclosed herein are set forth with particularity in the appended claims. A better understanding of the features and advantages of the compositions, methods, and articles disclosed herein will be obtained by reference to the following detailed description that sets forth illustrative embodiments, and the accompanying drawings (also “Figure” and “FIG.” herein), of which:

FIGS. 1A-1C provide a comparison of the active sites of butyrylcholinesterase (BChE) and cocaine esterase (CocE). FIG. 1A illustrates the catalytic triad active site of acidic amino acids (Asp 259, His 287, and Ser 117) of CocE (carbons and backbone in green); FIG. 1B illustrates the catalytic triad active site of acidic amino acids (Glu 325, His 438, and Ser 198) of BChE (carbons and backbone in purple); and FIG. 1C illustrates an overlay of CocE and BChE active sites showing structural conservation of functional groups of catalytic triad side chains.

FIGS. 2A-2C provide a simplistic docking of the organophosphate sarin into the active site of butyrylcholinesterase (BChE) and cocaine esterase (CocE). FIG. 2A illustrates a molecular structure of Sarin; FIG. 2B illustrates cocaine esterase with sarin modeled into the active site; and FIG. 2C illustrates butyrylcholinesterase with sarin modeled into the active site.

FIG. 3 illustrates a flowchart showing the steps of a process for manufacturing a CocE composition, according to an embodiment of the present invention.

FIGS. 4A and 4B illustrate a flowchart showing exemplary steps of a process for manufacturing CocE compositions by formulating recombinant CocE into a CocE composition (FIG. 4A) and a flowchart showing exemplary steps of a process for manufacturing CocE compositions by formulating a pharmaceutically acceptable excipient into the CocE composition (FIG. 4B).

FIG. 5 illustrate dichlorvos docked to CocE, with phosphate group of the ligand oriented toward the catalytic triad. Protein backbone is displayed in cartoon, dichlorvos ligand and protein side chains are displayed as sticks. All carbon atoms are colored black, and all heteroatoms are shaded gray.

FIG. 6 illustrates paraoxon docked to cocaine esterase, with phosphate group of the ligand oriented toward the catalytic triad. Protein backbone is displayed in cartoon, paraoxon ligand and protein side chains are displayed as sticks. All carbon atoms are colored black, and all heteroatoms are shaded gray.

FIG. 7 illustrates naled docked to cocaine esterase, with phosphate group of the ligand oriented toward the catalytic triad. Protein backbone is displayed in cartoon, naled ligand and protein side chains are displayed as sticks. All carbon atoms are colored black, and all heteroatoms are shaded gray.

FIG. 8 illustrates sarin docked to cocaine esterase, with phosphate group of the ligand oriented toward the catalytic triad and the propyl group nestled in a small hydrophobic cleft adjacent to the active site. Protein backbone is displayed in cartoon, sarin ligand and protein side chains are displayed as sticks. All carbon atoms are colored black, and all heteroatoms are shaded gray.

FIG. 9A is a mass spectrum illustrating the disappearance of dichlorvos as a substrate of cocaine esterase over 6 hours. FIG. 9B is a mass spectrum illustrating the disappearance of dichlorvos expressed as a percentage of original (control) amount over 360 minutes.

FIG. 10A is a mass spectrum illustrating the disappearance of cocaine as a substrate of cocaine esterase over 6 hours. FIG. 10B is a mass spectrum illustrating the disappearance of cocaine expressed as a percentage of original (control) amount over 360 minutes.

FIG. 11 illustrates an accessory configure to a respirator, wherein the accessory comprises a CocE, wherein 501 is a strap; 502 is a respirator body; 503 is a filter cartridge container; and 504 is a container of CocE.

FIG. 12 illustrates a vehicle (601) equipped with an air intake system comprising CocE (602).

### DETAILED DESCRIPTION

While various embodiments of the compositions, methods, and articles have been shown and described herein, it will be obvious to those skilled in the art that such embodiments are provided by way of example only. Numerous variations, changes, and substitutions may occur to those skilled in the art without departing from the compositions, methods, and articles disclosed herein. It should be understood that various alternatives to the embodiments of the compositions, methods, and articles described herein may be employed.

Despite decades-long bans on their use, the proliferation of organophosphate (OP) based chemical warfare agents (CWAs) persists. In recent years these chemicals have been used to terrorize populations, silence dissidents, and legally as pesticides where local communities may accidentally be poisoned. The use of organophosphate-based agents in these situations continues due to the ease of synthesis and straightforward deployment methods. If a well-tolerated, efficacious therapeutic were available, risks associated with this type of poisoning could be mitigated and potentially eliminated.

Chemical warfare agents (CWAs) inhibit acetylcholinesterase (AChE), and the subsequent dysregulation of acetylcholine signaling leads to an accumulation of a neurotransmitter, acetylcholine, causing toxicity in the exposed individual. Multiple pharmaceutical and biotechnological approaches have shown modest efficacy in combating these dangerous molecules. For instance, small molecules, such as, atropine, has been administered to ameliorate the effects of a build-up of acetylcholine by blocking acetylcholine receptors on neurons (J. N. Moore, Ratification of the Geneva Protocol on Gas and Bacteriological Warfare: A Legal and Political Analysis. Virginia Law Review 58, 419-509 (1972)). In addition, long-term central nervous system injury caused by OP-induced convulsion was ameliorated by the administration of an anticonvulsant, such as, benzodiazepine (P. F. Walker. (Springer International Publishing, Cham, 2017), pp. 379-400; R. Pita, J. Domingo, The Use of Chemical Weapons in the Syrian Conflict. Toxics 2, 391-402 (2014). However, none of these approaches are ideal, as in the case of atropine and benzodiazepine, symptoms are treated without affecting the cause.

An approach to combatting the cause rather than merely the symptoms is the use of butyrylcholinesterase (BChE), an 85 kDa protein. BChE has been used as a scavenger of nerve agents by binding and sequestering the toxins in a surmountable manner, one toxin to one protein. Meanwhile, oxime reactivators can be used to assist the esterase enzymes in completing the catalytic cycle by modestly increasing the rate of esterase-mediated hydrolysis. Recently, BChE has been engineered to enzymatically and processively hydrolyze CWAs into biologically inert molecules ((United States Department of State, state.gov, 2021), vol. 2021). However, it is difficult produce BChE at scale, and coupling the enzyme with oxime reactivators remain prohibitively more expensive as at least 200 mg of BChE is required to treat a 70 kg person with exposure to 2 LD50 of nerve agent (D. A. Shea. (Congressional Research Service, the Library of Congress, 2013)).

In some embodiments, disclosed herein is a novel use for an enzyme that has a demonstrated record of safety and efficacy. The discovery of a cost-effective therapeutic for organophosphate poisoning, having thermostability properties and remaining active despite exposure to rugged environments, can prove to be transformational in the effort to mitigate risks posed by organophosphate poisoning. The use of bacterially derived hydrolases, e.g., phosphotriesterases (PTEs), for the detoxification of chemical CWAs has been attempted in the past; however, none of these engineered, metal-dependent phosphotriesterase enzymes have yielded a medical counter measure (MCM) that can serve as an antidote against highly toxic CWAs. In addition to poor retention after intravenous injection, injection of PTEs have resulted in immune activation (S. Costanzi, J.-H. Machado, M. Mitchell, Nerve Agents: What They Are, How They Work, How to Counter Them. ACS Chemical Neuroscience 9, 873-885 (2018)). Metal-independent cocaine esterase (CocE) suffered from similar immune activation in early primate studies, however, throughout clinical trials where CocE (under the brand name RBP-8000) was administered via intravenous injection, no induction of immune response was detected (A. S. Cornelissen, S. D. Klaassen, T. van Groningen, S. Bohnert, M. J. A. Joosen, Comparative physiology and efficacy of atropine and scopolamine in sarin nerve agent poisoning. Toxicology and Applied Pharmacology 396, 114994 (2020); T. C. Marrs, Diazepam in the Treatment of Organophosphorus Ester Pesticide Poisoning. Toxicological Reviews 22, 75-81 (2003); R. Kuruba, X. Wu, D. S. Reddy, Benzodiazepine-refractory status epilepticus, neuroinflammation, and interneuron neurodegeneration after acute organophosphate intoxication. Biochimica et Biophysica Acta (BBA)-Molecular Basis of Disease 1864, 2845-2858 (2018)). CocE, as well as the inclusion of oxime derivatives, can serve as a medical counter measure for CWAs. Rationally and computationally designed CocE mutants can optimize properties of the CocE enzyme for use as a treatment for OP toxicity. In some embodiment, the compositions, methods, and articles disclosed herein relate to the ability of CocE to enzymatically and processively hydrolyze CWAs in vitro, potentially with the assistance of oximes. A related enzyme, butyrylcholinesterase (BChE), is able to at a minimum stoichiometrically scavenge CWAs to act as an antidote and recently a mutant form of BChE has demonstrated an ability to act catalytically to degrade CWAs. Recombinant CocE is produced through fermentation of bacteria allowing for large scale, relatively inexpensive expression of the enzyme. Engineered mutants of CocE are thermostable and have been formulated to be successfully used as test material for phase I and phase II clinical trials in humans. When tested head-to-head against certain substrates of CocE and BChe, CocE has been shown to have a similar Vₘₐₓ as engineered BChE. CocE is safe and less expensive to produce and can be the medical counter measure of choice for CWA intoxication.

### Chemical Weapons

As define herein, the term chemical weapon includes any of the substances defined in Article II of the Chemical Weapons Convention. These include toxic chemicals and their precursors, munitions and devices, specifically designed to cause death or other harm through the toxic properties of those defined toxic chemicals, which would be released as a result of the employment of such munitions and devices; and any equipment specifically designed for use directly in connection with the employment of such munitions and devices.

In some embodiments, the “toxic chemicals” are any chemical which through its chemical action on life processes can cause death, temporary incapacitation or permanent harm to humans or animals, regardless of their origin or of their method of production, and regardless of whether they are produced in facilities, in munitions or else.

In some embodiments, the “precursors” are any chemical reactant which takes part at any stage in the production by whatever method of a toxic chemical. It includes any key component of a binary or multicomponent chemical system. Herein, the key component of binary or multicomponent chemical systems means the precursor which plays the most important role in determining the toxic properties of their final product and reacts rapidly with other chemicals in the binary or multicomponent system.

In some embodiments, a chemical weapon is a toxic chemical that is used to cause intentional harm or death. In some embodiments, a chemical weapon is a toxic chemical contained in a delivery system, such as, bomb or artillery shell. Under this definition, munitions, devices, and other equipment designed to weaponize toxic chemicals fall under a chemical weapon.

In some embodiments, munitions or devices are specifically designed to cause harm or death through the release of toxic chemicals. Examples of munitions or devices include, but not limited to, mortars, artillery shells, missiles, bombs, or spray tanks.

Examples of chemical weapons disclosed herein include, but not limited to, (a) fully-developed chemical weapons and the components of such weapons when stored separately (e.g., binary munitions); (b) chemicals used to produce chemical weapons (e.g., precursors); (c) chemicals used to cause intentional death or harm; (d) items with peaceful civilian uses, when used or intended for chemical weapons use (e.g., dual-use items); (e) munitions and devices intended for the delivery of toxic chemicals; and (f) equipment directly in connection with aforementioned munitions and devices.

Upon accidental exposure to these chemical weapons, a range of symptoms, including incontinence, pinpoint pupils, chest tightness, shortness of breath, nausea, vomiting, runny nose, excessive salivation and sweating, abdominal cramps, muscle twitching, confusion, seizures, paralysis, coma, respiratory paralysis, and death, may occur. Incapacitating and fatal effects may occur within minutes to hours.

Examples of chemical weapons include, but are not limited to, nerve agents comprising organophosphate-based nerve agents and organophosphate pesticides. Organophosphate-based nerve agents act rapidly and highly toxic. Organophosphate-based pesticides exhibit the same physiological reaction as organophosphate-based nerve agents and are considered extremely poisonous. Organophosphate-based pesticides include bomyl, carbophenothion, chlorfenvinphos, chlormephos, chlorthiophos, cyanofenphos, dialifor, dicrotophos, dimefos, dioxathion, disulfoton, endothion, EPN, ethyl parathion, famphur, fenamiphos, fenophosphon, fensulfothion, fonofos, fosthietan, isofenphos, mephosfolan, methamidophos, mevinphos, mipafox, monocrotophos, phosfolan, phosphamidon, prothoate, schradan, and tetraethyl pyrophosphate.

### Nerve Agents

As a chemical weapon, nerve agents, also called nerve gases, are a class of organic chemicals that disrupt the mechanism by which nerves transfer messages to organs. In some embodiments, the nerve agents are acetylcholinesterase (AchE) inhibitors, by which AChE, an enzyme that catalyzes the breakdown acetylcholine, a neurotransmitter, is blocked.

Nerve agents can be potent, irreversible AChE inhibitors that cause rapid onset of severe clinical effects up to and including death. Though effective to some extent, currently available medical countermeasures (MCMs) (e.g., atropine, 2-PAM, and diazepam) are not without limitations and do not effectively prevent or ameliorate all the adverse effects of nerve agent intoxication. Because it is not ethical or feasible to evaluate the efficacy of new MCMs for nerve agent intoxication in humans, candidate MCMs are typically evaluated using in vitro or animal models. For MCMs that bind or degrade nerve agents in vivo, thus removing them from the circulation, effectiveness may be evaluated in vitro by measuring the effects of the MCM on nerve agent concentration in solution over time. A complementary approach is to evaluate protection of AChE activity from inhibition by CWNAs in vitro in the presence and absence of the MCM.

Symptoms of nerve agent poisoning include, but not limited to, constriction of pupils, profuse salivation, convulsions, and involuntary urination, and defecation. The first symptoms appear in seconds after exposure, and the loss of the body's control over respiratory and other muscles may lead to death by asphyxiation or cardiac arrest in minutes.

In some embodiments, the nerve agents are readily vaporized or aerosolized, and thus the respiratory system is the primary portal of entry into the body. In some embodiments, the nerve agents are absolved through the skin, requiring a person exposed to such agents to wear a full body protective suit along with a respirator. In some embodiments, existing methods for spreading nerve agents include, but are not limited to, uncontrolled aerosol munitions, smoke generation, explosive dissemination, atomizers, humidifies, and foggers. The choice of such spread methods may depend on the physical properties of the nerve agents used, the nature of the target, the achievable level of sophistication, etc.

In some embodiments, existing nerve agents may be colorless to amber-colored (or a pure chroma color), tasteless liquids that may evaporate to a gas. In some embodiments, existing agents (e.g., Sarin and VX) may be odorless. In some embodiments, existing nerve agents (e.g., Tabun) may possess a slightly fruity odor. In some embodiments, existing nerve agents (e.g., Soman) may possess a slight camphor odor.

### Organophosphate-Based Nerve Agent

In some embodiments, examples of organophosphate-based nerve agent include, but are not limited to G-series nerve agents (e.g., tabun (GA), sarin (GB), soman (GD), cyclosarin (GF)), V-series nerve agents (e.g., VE, VG, VM, VP, VR, VS, VS, VX, Chinese VX, Russian VX, EA-3148, and EA-2192), GV-series nerve agents (GV and 2-dimethylaminoethyl-(dimethylamido)-fluorophosphate), carbamates (e.g., aldicarb, methomyl, EA-3990, and EA-4056), and Fourth generation agents, also known as Novichoks, A-series nerve agents (e.g. substance-33, A230, A232, A234, Novichok-5, and Novichok-7), or a combination thereof.

### Organophosphate Pesticides

Organophosphate pesticides and organophosphate insecticides are chemicals that poison insects and mammals. Such chemicals are used in agriculture, the home, gardens, and veterinary practice. Organophosphate pesticides damage an enzyme in the body, e.g., acetylcholinesterase, which is critical for controlling never signals in the body. Although this damage to the enzyme kills pests, it may also cause undesired side effects in exposed humans. Similarly, the organophosphate-based compounds are widely used as pesticides which cause a threat to human health. Organophosphate pesticides are thiols, amides, or esters of phosphonic, phosphinic, phosphoric, or thiophosphoric acids with two additional organic side chains of the phenoxy, cyanide, or thiocyanate group. Some of the organophosphate pesticides belong to the phosphonothioates (S-substituted), phosphonofluoridate categories comprise of nerve agents, commonly known as chemical warfare agents.

In some embodiments, examples of organophosphate insecticides include, but are not limited to azamethiphos, azinphos methyl, bomyl, carbamates (e.g., aldicarb, methomyl, EA-3990, and EA-4056), carbophenothion, chlorethoxyphos, chlorfenvinphos, chlormephos, chlorpyrifos, chlorpyrifos methyl, chlorthiophos, coumaphos, cyanofenphos, demeton, dialifor, dialkylphosphates (DAPs), diazinon, dichlorvos, dicrotophos, diethyldithiophosphate (DEDTP), diethylphosphate (DEP), dimefos, dimefox, dimethoate, dimethyldithiophosphate (DMDTP), dimethylthiophosphate (DMTP), dioxathion, disulfoton, endothion, EPN, ethion, ethyl parathion, famphur, fenamiphos, fenophosphon, fensulfothion, fenthion, fenitrothion, fonofos, fosthietan, isofenphos, 2-isopropyl-4-methyl-6-hydroxypyrimidine (IMPY), isazophos methyl, malathion, mephosfolan methamidophos, methidathion, methyl parathion, mevinphos, mipafox, monocrotophos, oxydemeton methyl, parathion (or ethyl parathion), paraoxon, phorate, phosfolan, phosmet (or imidan), phosphamidon, pirimiphos methyl, prothoate, schradan, sulfotepp, temephos, terbuos, tetrachlorvinphos, tetraethyl pyrophosphate 3,5,6-trichloro-2-pyridinol (TCPy), dimethyl 1,2-dibromo-2,2-dichloroethylphosphate (or naled or dibrom), or a combination thereof.

### Cocaine Esterase

While BChE performs a variety of endogenous functions in a human with no exposure to toxins, it is also the body's primary enzyme to facilitate clearance of cocaine upon the drug's ingestion. In attempts to create an antidote for acute cocaine toxicity, BChE has been engineered to hydrolyze cocaine more efficiently (D. M. Cerasoli et al., (07) In vitro and in vivo characterization of recombinant human butyrylcholinesterase (Protexia™) as a potential nerve agent bioscavenger. Chemico-Biological Interactions 157-158, 362-365 (2005)). In a quirk of evolution, a species of Rhodococcus has evolved in an environment where cocaine was abundant and developed the ability to utilize cocaine as a carbon and nitrogen source. The first step in the pathway of cocaine metabolism by the bacteria is to use a 62 kDa enzyme, known as “Cocaine Esterase” (CocE), to hydrolyze a molecule of cocaine into the exactly same molecular species that BChE hydrolyses cocaine into in a human. In some embodiments, CocE detoxifies an organophosphate-based agent by hydrolysis, that is, the breakdown of cocaine into metabolite ecgonine methyl ester and benzoic acid (N. Aurbek, H. Thiermann, F. Eyer, P. Eyer, F. Worek, Suitability of human butyrylcholinesterase as therapeutic marker and pseudo catalytic scavenger in organophosphate poisoning: A kinetic analysis. Toxicology 259, 133-139 (2009)). CocE has been engineered into a highly active, thermostable enzyme through a series of mutations that, when PEGylated, can be safely administered to humans and is efficacious in rapidly reducing plasma concentrations of non-hydrolyzed cocaine (T.-M. Shih, J. A. Guarisco, T. M. Myers, R. K. Kan, J. H. McDonough. The oxime pro-2-PAM provides minimal protection against the CNS effects of the nerve agents sarin, cyclosarin, and VX in guinea pigs. Toxicology Mechanisms and Methods 21, 53-62 (2011); R. K. Sit et al., New Structural Scaffolds for Centrally Acting Oxime Reactivators of Phosphylated Cholinesterases *. Journal of Biological Chemistry 286, 19422-19430 (2011)). As opposed to the expensive and time-consuming generation of engineered BChE in transfected human cells, engineered CocE can be readily expressed in large quantities by fermentation of e. coli.

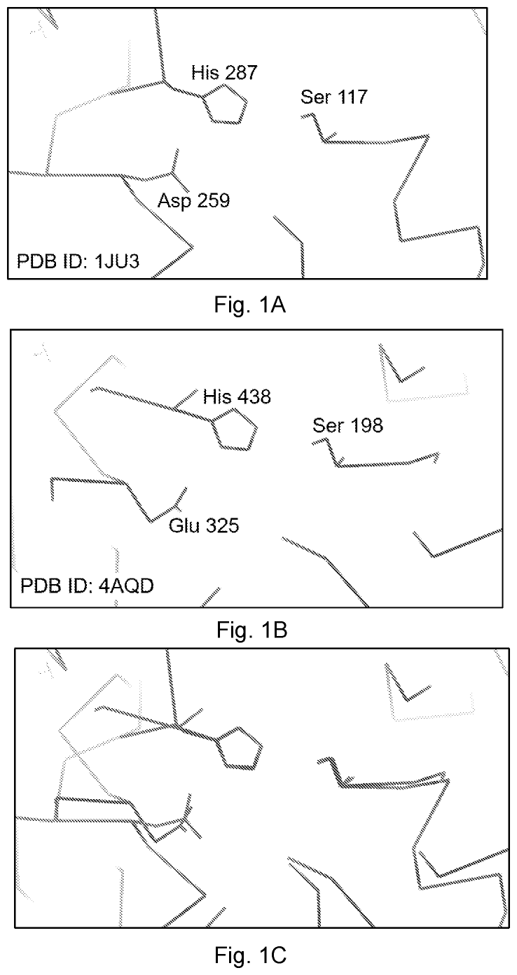

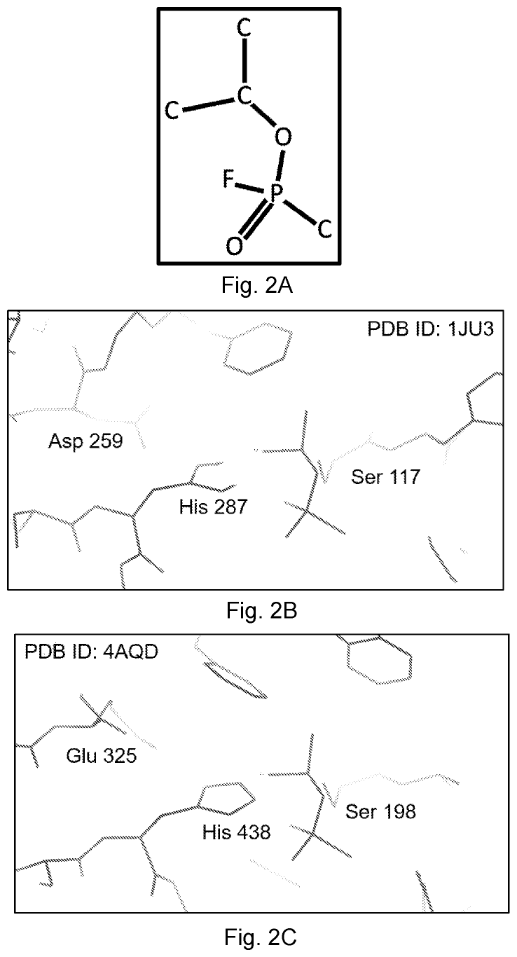

Although BChE has been purified from human plasma, human embryonic kidney cells, mammalian milk, and insect cells to obtain protein that can protect against both cocaine and organophosphate toxicity ((United States Department of State, state.gov, 2021), vol. 2021; D. A. Shea. (Congressional Research Service, the Library of Congress, 2013); R. K. Sit et al., Imidazole Aldoximes Effective in Assisting Butyrylcholinesterase Catalysis of Organophosphate Detoxification. Journal of Medicinal Chemistry 57, 1378-1389 (2014); K. G. McGarry et al., A Novel, Modified Human Butyrylcholinesterase Catalytically Degrades the Chemical Warfare Nerve Agent, Sarin. Toxicol Sci 174, 133-146 (2020)), each of these methods was time and resource intensive. In one attempt, both BChE and CocE have been engineered to be efficient and to have comparable Vₘₐₓ when cocaine is the substrate (X. Brazzolotto et al., Human butyrylcholinesterase produced in insect cells: huprine-based affinity purification and crystal structure. The FEBS Journal 279, 2905-2916 (2012)). Comparison of the active sites of BChE (PDB ID 4AQD) and CocE (PDB ID 1JU3) revealed that both possess the classical catalytic triad of an acidic amino acid (aspartate or glutamate), histidine and serine, indicating a shared enzymatic mechanism (FIGS. 1A-1C) (D. A. Shea. (Congressional Research Service, the Library of Congress, 2013); X. Brazzolotto et al., Human butyrylcholinesterase produced in insect cells: huprine-based affinity purification and crystal structure. The FEBS Journal 279, 2905-2916 (2012); C.-G. Zhan, F. Zheng, D. W. Landry, Fundamental Reaction Mechanism for Cocaine Hydrolysis in Human Butyrylcholinesterase. Journal of the American Chemical Society 125, 2462-2474 (2003)). Additionally, simplistic docking of the organophosphate sarin into the active site of each esterase showed a pocket capable of fitting the nerve agent, which is much smaller than the cocaine substrate (FIGS. 2A-2C). The molecular mechanism that allowed human BChE engineered with four mutations (Y282N, G283H, T284M, and P285L) to processively hydrolyze nerve agents when wild type BChE could not, remained unclear ((United States Department of State, state.gov, 2021), vol. 2021). Despite an active site that superimposes the functional groups of the catalytic triad residues with nearly perfect fidelity, the pathways from outside the confines of the enzyme into the active site and the local dynamics were quite different between the two enzymes (D. A. Shea. (Congressional Research Service, the Library of Congress, 2013); N. Aurbek, H. Thiermann, F. Eyer, P. Eyer, F. Worek, Suitability of human butyrylcholinesterase as therapeutic marker and pseudo catalytic scavenger in organophosphate poisoning: A kinetic analysis. Toxicology 259, 133-139 (2009)). Given the mechanistic similarities, disclosed invention herein are compositions, methods, and articles relating to the use of CocE for treatment in the event of organophosphate exposure.

In some embodiments, the CocE is an enzyme that can detoxify a chemical weapon by the breakdown of organophosphate-based agents. In some embodiments, the CocE is an enzyme having 62 kDa. In some embodiments, the CocE is encoded by a nucleic acid sequence provided in SEQ ID NO:1 (Table 1). In some embodiments, the CocE is encoded by a nucleic acid sequence that is at least about 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to any one of SEQ ID NO: 1-3.

TABLE 1
CocE Sequences

| SEQ. | | |
|---|---|---|
| ID | Sequence | Description |
| 1 | MVDCNYSVAC NVMVPMRDGV RLAVDLYRPD ADGPVPVLLV | PDB: 312F |
| | RNPYDKFDVF AWSTQSTNWL EFVRDGYAVV IQDTRGLFAS | with G4C, |
| | EGEFVPHVDD ECDAEDTLSW ILEQAWCDGN VGMFGVSYLG | S10C, |
| | VTQWQAAVSG VGGLKAIAPS MASADLYRAP WYGPGGALSV | S288T, |
| | EALLGWSALI GRQLITSRCD ARPEDAADFV QLAAILNDVA GAASVTPLAE | A92C, and |
| | QPLLGRLIPW VIDQVVDHPD NDESWQSISL FERLGGLATP ALITAGWYDG | S179C |
| | FVGESLRTFV AVKDNADARL VVGPWSHTNL TGRNADRKFG | |
| | IAATYPIQEA TTMHKAFFDR HLRGETDALA GVPKVRLFVM GIDEWRDETD | |
| | WPLPDTAYTP FYLGGSGAAN TSTGGGTLST SISGTESADT YLYDPADPVP | |
| | SLGGTLLFHN GDNGPADQRP IHDRDDVLCY STEVLTDPVE VTGTVSARLF | |
| | VSSSAVDTDF TAKLVDVFPD GRAIALCDGI VRMRYRETLV NPTLIEAGEI | |
| | YEVAIDMLAT SNVFLPGHRI MVQVSSSNFP KYDRNSNTGG VIAREQLEEM | |
| | CTAVNRIHRG PEHPSHIVLP IIKRKLAAAL E | |
| 2 | MVDGNYSVASNVMVPMRDGVR LAVDLYRPDADGPVPVLLVRNPY | PDB: |
| | DKFDVFAWSTQSTNWLEFVRDG YAVVIQDTRGLFASEGEFVPHVD | 3I2J_A |
| | DEADAEDTLSWILEQAWCDGNV GMFGVSYLGVTQWQAAVSGVG | |
| | GLKAIAPSMASADLYRAPWYGPG GALSVEALLGWSALIGTGLITSRS | |
| | DARPEDAADFVQLAAILNDVAG AASVTPLAEQPLLGRLIPWVIDQ | |
| | VVDHPDNDESWQSISLFERLGGL ATPALITAGWYDGFVGESLRTF | |
| | VAVKDNADARLVVGPWSHSNLT GRNADRKFGIAATYPIQEATTMH | |
| | KAFFDRHLRGETDALAGVPKVR LFVMGIDEWRDETDWPLPDTAY | |
| | TPFYLGGSGAANTSTGGGTLSTS ISGTESADTYLYDPADPVPSLGG | |
| | TLLFHNGDNGPADQRPIHDRDD VLCYSTEVLTDPVEVTGTVSARL | |
| | FVSSSAVDTDFTAKLVDVFPDGR AIALCDGIVRMR YRETL VNPTLIE | |
| | AGEIYEVAIDMLATSNVFLPGHRI MVQVSSSNFPKYDRNSNTGGV | |
| | IAREQLEEMCTA VNRIHR GPEHPSHIVLPIIKRKLAAALEHHHHHH | |
| 3 | MVDGNYSVASNVMVPMRDGVRL AVDLYRPDADGPVPVLLVRNPYD | PDB: 3I2F |
| | KFDVFAWSTQSTNWLEFVRDGYA VVIQDTRGLFASEGEFVPHVD | |
| | DEADAEDTLSWILEQAWCDGNVGMFG VSYLGVTQWQAAVSGVGGL | |
| | KAIAPSMASADLYRAPWYGPGGALSVEALLGWSALIGRQLITSRSDARPEDA | |
| | ADFVQLAAILNDVAGAASVTPLAE QPLLGRLIPWVIDQVVDHPDNDES | |
| | WQSISLFERLGGLATPALITAGWYD GFVGESLRTFVAVKDNADARLVVG | |
| | PWSHSNLTGRNADRKFGIAATYPIQ EATTMHKAFFDRHLRGETDALAGV | |
| | PKVRLFVMGIDEWRDETDWPLPDT AYTPFYLGGSGAANTSTGGGTLST | |
| | SISGTESADTYLYDPADPVPSLGGT LLFHNGDNGPADQRPIHDRDDVLC | |
| | YSTEVLTDPVEVTGTVSARLFVSSS AVDTDFTAKLVDVFPDGRAIALCD | |
| | GIVRMRYRETLVNPTLIEAGEIYEV AIDMLATSNVFLPGHRIMVQVSSSN | |
| | FPKYDRNSNTGGVIAREQLEEMCTA VNRIHRGPEHPSHIVLPIIKRKLAAAL | |
| | EHHHHHH | |

In some embodiments, the CocE is a recombinant protein enzyme that can detoxify a chemical weapon by the breakdown of organophosphate-based agents. In some embodiments, the recombinant protein enzyme of CocE is produced through recombinant DNA (rDNA) technology in a non-disease-producing strain of E. coli bacteria.

In some embodiments, the CocE comprises at least two mutations. In some embodiments, the CocE comprises at least two mutations, comprising T172R and G173Q.

In some embodiments, the CocE comprises a catalytic triad of aspartate, histidine, and serine. In some embodiments, the CocE comprises a catalytic triad of glutamate, histidine, and serine.

Functionalization of CocE with Hydrophilic Polymer

Although protein and peptide drugs show promise as therapeutic agents, many of them are thermo-instable and degraded by circulating proteases, may be rapidly cleared by the kidneys, generate neutralizing antibodies and immunogenicity, and have a short circulating half-life. Likewise, administration of CocE has been considered as a promising approach to treat cocaine overdose and addiction, but such issues have been obstacles to the clinical application of CocE. To address these issues, CocE may be conjugated with a hydrophilic polymer.

In some embodiments, the hydrophilic polymer may comprise a linear polymer or may be a branched polymer. For instance, the hydrophilic polymer comprises a molecule selected from the group consisting of poly(ethylene glycol) (PEG, also known as poly(ethylene oxide) (PEO) or polyoxyethylene), poly(vinyl alcohol) (PVA), poly(vinyl pyridine), poly(vinyl pyrrolidone) (PVP), poly(acrylic acid) (PAA), polyacrylamide, poly(N-isopropylacrylamide) (PNIPAM), poly(methyl methacrylate) (PMMA), poly(2-hydroxylethyl methacrylate) (PHEMA), poly(oligo (ethylene glycol) methyl ether methacrylate) (POEGMA), poly(glutamic acid) (PGA), poly-lysine, poly-glucoside, streptavidin, and dextran.

Examples of suitable branched polymers include, but are not limited to, branched PEG, branched poly(vinyl alcohol) (branched PVA), branched poly(vinyl pyridine), branched poly(vinyl pyrrolidone) (branched PVP), poly(acrylic acid) (branched PAA), branched polyacrylamide, branched poly(N-isopropylacrylamide) (branched PNIPAM), branched poly(methyl methacrylate) (branched PMMA), branched poly(2-hydroxylethyl methacrylate) (branched PHEMA), branched poly(oligo (ethylene glycol) methyl ether methacrylate) (branched POEGMA), branched polyglutamic acid (branched PGA), branched poly-lysine, branched poly-glucoside, and dextran.

In some instances, the branched polymers disclosed herein may comprise at least 4 branches, at least 5 branches, at least 6 branches, at least 7 branches, at least 8 branches, at least 9 branches, at least 10 branches, at least 12 branches, at least 14 branches, at least 16 branches, at least 18 branches, at least 20 branches, at least 22 branches, at least 24 branches, at least 26 branches, at least 28 branches, at least 30 branches, at least 32 branches, at least 34 branches, at least 36 branches, at least 38 branches, or at least 40 branches. Molecules often exhibit a ‘power of 2’ number of branches, such as 2, 4, 8, 16, 32, 64, or 128 branches.

### PEGylation of CocE

Particularly, covalent and non-covalent attachment of a PEG molecule to biological molecules, such as proteins and enzymes, such as CocE, called PEGylation, can improve pharmacokinetics of such biological molecules by increasing the molecular mass of proteins and peptides and shielding them from proteases. Each PEG segment can combine with two or three molecules, making the overall compound larger and more hydrophilic. The PEG structure may be either linear or branched, and the branched PEG tends to increase in vivo half-life by increasing “stealth” properties of a conjugated biological molecules. Also, PEGylation allows to modify physiological properties and prolong the retention of the therapeutic agents in the body.

The introduction of different functional groups to the end of a PEG molecule allows for more site-specific reactions. For instance, various amino acid residue in proteins may get involved in chemical reactions with PEG having amine, sulfhydryl, carboxyl, and carbonyl groups. By altering the chain-end functional group of the PEG molecules, it may get easier to target these amino acid residues or specific functional groups. Examples of chain-end functional groups include, but not limited to, carboxyl, N-hydroxysuccinimide (NHS), anhydride, ester, aminooxy, amino, alkyne, azide, bicyclo[6.1.0] nonyne (BCN), dibenzocyclooctyne (DBCO), trans-cyclooctene (TCO), tetrazine, bromo, or a combination thereof.

In some embodiments, molecular weight of linear PEG ranges from about 100 to about 1,000,000 g/mol. In some embodiments, molecular weight of linear PEG ranges from about 100 to about 800,000 g/mol. In some embodiments, molecular weight of linear PEG ranges from about 100 to about 500,000 g/mol. In some embodiments, molecular weight of linear PEG ranges from about 100 to about 100,000 g/mol. In some embodiments, molecular weight of linear PEG ranges from about 100 to about 50,000 g/mol. In some embodiments, molecular weight of linear PEG ranges from about 100 to about 40,000 g/mol. In some embodiments, molecular weight of linear PEG ranges from about 100 to about 30,000 g/mol. In some embodiments, molecular weight of linear PEG ranges from about 500 to about 30,000 g/mol. In some embodiments, molecular weight of linear PEG ranges from about 1,000 to about 30,000 g/mol. In some embodiments, molecular weight of linear PEG ranges from about 2,000 to about 30,000 g/mol. In some embodiments, molecular weight of linear PEG ranges from about 3,000 to about 30,000 g/mol. In some embodiments, molecular weight of linear PEG ranges from about 4,000 to about 30,000 g/mol. In some embodiments, molecular weight of linear PEG ranges from about 4,000 to about 25,000 g/mol. In some embodiments, molecular weight of linear PEG ranges from about 5,000 to about 25,000 g/mol. In some embodiments, molecular weight of linear PEG ranges from about 5,000 to about 20,000 g/mol. In some embodiments, molecular weight of linear PEG ranges from about 100 to about 500 g/mol. In some embodiments, the desired molecular weight of linear PEG ranges from about 5,000 to about 20,000 g/mol.

In some embodiments, molecular weight of branched PEG ranges from about 100 to about 1,000,000 g/mol. In some embodiments, molecular weight of branched PEG ranges from about 1,000 to about 500,000 g/mol. In some embodiments, molecular weight of branched PEG ranges from about 5,000 to about 100,000 g/mol. In some embodiments, molecular weight of branched PEG ranges from about 10,000 to about 80,000 g/mol. In some embodiments, molecular weight of branched PEG ranges from about 12,000 to about 70,000 g/mol. In some embodiments, molecular weight of branched PEG ranges from about 13,000 to about 60,000 g/mol. In some embodiments, molecular weight of branched PEG ranges from about 14,000 to about 55,000 g/mol. In some embodiments, molecular weight of branched PEG ranges from about 15,000 to about 50,000 g/mol. In some embodiments, molecular weight of branched PEG ranges from about 16,000 to about 48,000 g/mol. In some embodiments, molecular weight of branched PEG ranges from about 17,000 to about 46,000 g/mol. In some embodiments, molecular weight of branched PEG ranges from about 18,000 to about 44,000 g/mol. In some embodiments, molecular weight of branched PEG ranges from about 19,000 to about 42,000 g/mol. In some embodiments, molecular weight of branched PEG ranges from about 20,000 to about 40,000 g/mol. In some embodiments, the desired molecular weight of branched PEG ranges from about 20,000 to about 40,000 g/mol.

### Oxime Compounds

Oxime compounds can be used to assist esterase enzymes, such as, cholinesterase, CocE, etc., in completing the catalytic cycles, increasing the rate of esterase-mediated hydrolysis. In the case of acetylcholinesterase, oximes work by removal of the phosphoryl group from the inhibited acetylcholinesterase enzyme, resulting in enzyme reactivation. In some embodiments, CocE binds an organophosphate-based agent and release the first product of hydrolysis, leaving a second product covalently bound to the active site serine, upon which an oxime is added to assist CocE to release the second product (or reactivate the CocE for a fresh catalytic cycle).

In some embodiments, the oxime compound disclosed herein is selected from the group consisting of pralidoxime (2-PAM), asoxime (HI-6), deazapralidoxime (DZP), methoxime (MMB4), obidoxime, trimedoxime (TMB4), TAB2OH, ortho-7, 3-hyroxy-2-pyridinealdoxime, a derivative thereof, or a combination thereof.

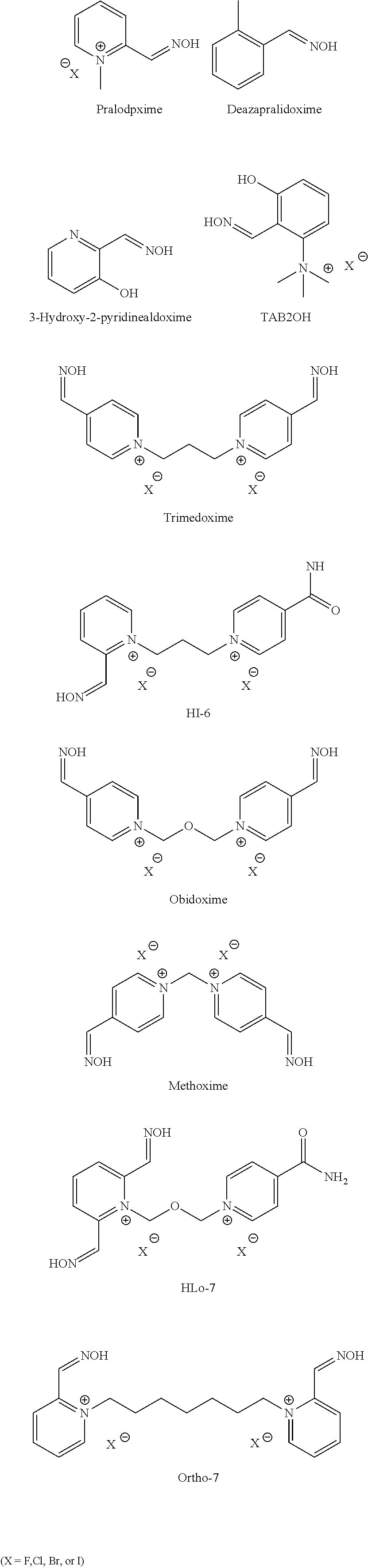

### Pharmaceutically Acceptable Excipients

The pharmaceutically acceptable excipients described herein refer to excipients which aid in the manufacturing or administration of the compositions described herein. Non-limiting examples of such excipient include solvents, flavorants, colorants, palatants, antioxidants, viscosity modifying agents, tonicity agents, drug carriers, sustained-release agents, comfort-enhancing agents, emulsifiers, solubilizing aids, lubricants, binding agents, stabilizing agents, and other agents to aid in the manufacturing or administration of the compositions. The excipients used in the present invention are acceptable for use in pharmaceutical or nutraceutical applications or as food ingredients. The amount of pharmaceutically acceptable excipients may vary depending on the concentration of cocaine esterase or oxime compound in the composition for detoxifying an organophosphate-based agent or for administration methods by oral intake, inhalation, injection, topical application, or a combination thereof. A discussion of pharmaceutically acceptable carriers/excipients can be found in Remington's Pharmaceutical Sciences, Gennaro, AR, ed., 20th edition, 2000: Williams and Wilkins PA, USA. Exemplary pharmaceutically acceptable carriers can include salts, for example, mineral acid salts such as hydrochlorides, hydrobromides, phosphates, sulfates, and the like; and the salts of organic acids such as acetates, propionates, malonates, benzoates, and the like. For example, compositions described herein may be provided in liquid form, and formulated in saline based aqueous solution of varying pH (5-8), with or without detergents such polysorbate-80 at 0.01-1%, or carbohydrate additives, such mannitol, sorbitol, or trehalose. Commonly used buffers include histidine, acetate, phosphate, or citrate.

### Aqueous Solvents

Aqueous, or water-based, solvents are commonly used as a pharmaceutically acceptable excipients due to their lack of toxicity and low cost. Based on purity, different types of water may be defined, including purified water, highly-purified water, water for injections, sterilized water for injections.

### Non-Aqueous Solvents

In subcutaneous or intramuscular pharmaceutical formulations, non-aqueous solvents have been used to dissolve water-insoluble biomaterials, including protein enzymes. In some instances, the non-aqueous solvents comprise acetic acid, acetone, anisole, 1-butanol, 2-butanol, butyl acetate, tert-butylmethyl ether, diglyme, dimethyl isosorbide, dimethyl sulfoxide, ethanol, ethyl acetate, ethyl lactate, ethyl ether, ethyl formate, formic acid, heptane, glycofurol, glycerol formal, isobutyl acetate, isopropyl acetate, methyl acetate, 3-methyl-1-butanol, methylethyl ketone, 2-methyl-1-propanol, N-methyl-2-pyrrolidone, pentane, 1-pentanol, 1-propanol, 2-propanol, propylene glycol, poly(ethylene glycol), propyl acetate, Solketal, triethylamine, tetrahydrofurfuryl alcohol, or a combination thereof.

### Compositions

In some embodiments, the present disclosure comprises a composition having, by weight, a molar excess of oxime and a cocaine esterase. In some embodiments, the compositions disclosed herein have a 10-fold molar excess of oxime and a cocaine esterase. In some embodiments, the compositions comprise a cocaine esterase of approximately 65,000 Daltons and an oxime ranging from about 135 Daltons to 230 Daltons. In some embodiments, a patient is administered a composition comprising a cocaine esterase and an oxime of approximately 200 Daltons, wherein the patient is administered about 200 mg of cocaine esterase and about 6 mg of oxime.

In some embodiments, the present disclosure comprises a composition having, by weight a molar excess of oxime and a cocaine esterase and a pharmaceutically acceptable excipient.

In some embodiments, the compositions disclosed herein relate to compositions comprising a cocaine esterase. In some embodiments, the compositions comprise a cocaine esterase and an oxime compound. In some embodiments, the compositions comprise a cocaine esterase, an oxime compound, and one or more pharmaceutically acceptable excipients. The compositions can include a specific amount of a cocaine esterase content, made up of a desired amount of a cocaine esterase, a desired amount of an oxime compound, and one or more pharmaceutically acceptable excipients.

The compositions disclosed herein can include a cocaine esterase in a specific amount. In some embodiments, the specific amount of cocaine esterase is accurate to one significant figure. In another embodiment, the specific amount of cocaine esterase is accurate to two, three or four significant figures. The presence of the cocaine esterase in a specific amount in the composition allows for the same desired specific amount of the cocaine esterase to be present in various batches of the composition.

In one embodiment, the specific dose of the cocaine esterase ranges from about 50 mg to about 400 mg. In some embodiments, the specific dose of the cocaine esterase ranges from about 50 mg to about 80 mg, about 50 mg to about 110 mg, about 50 mg to about 140 mg, about 50 mg to about 170 mg, about 50 mg to about 210 mg, about 50 mg to about 240 mg, about 50 mg to about 270 mg, about 50 mg to about 300 mg, about 50 mg to about 330 mg, about 50 mg to about 360 mg, about 50 mg to about 400 mg, about 80 mg to about 110 mg, about 80 mg to about 140 mg, about 80 mg to about 170 mg, about 80 mg to about 210 mg, about 80 mg to about 240 mg, about 80 mg to about 270 mg, about 80 mg to about 300 mg, about 80 mg to about 330 mg, about 80 mg to about 360 mg, about 80 mg to about 400 mg, about 110 mg to about 140 mg, about 110 mg to about 170 mg, about 110 mg to about 210 mg, about 110 mg to about 240 mg, about 110 mg to about 270 mg, about 110 mg to about 300 mg, about 110 mg to about 330 mg, about 110 mg to about 360 mg, about 110 mg to about 400 mg, about 140 mg to about 170 mg, about 140 mg to about 210 mg, about 140 mg to about 240 mg, about 140 mg to about 270 mg, about 140 mg to about 300 mg, about 140 mg to about 330 mg, about 140 mg to about 360 mg, about 140 mg to about 400 mg, about 170 mg to about 210 mg, about 170 mg to about 240 mg, about 170 mg to about 270 mg, about 170 mg to about 300 mg, about 170 mg to about 330 mg, about 170 mg to about 360 mg, about 170 mg to about 400 mg, about 210 mg to about 240 mg, about 210 mg to about 270 mg, about 210 mg to about 300 mg, about 210 mg to about 330 mg, about 210 mg to about 360 mg, about 210 mg to about 400 mg, about 240 mg to about 270 mg, about 240 mg to about 300 mg, about 240 mg to about 330 mg, about 240 mg to about 360 mg, about 240 mg to about 400 mg, about 270 mg to about 300 mg, about 270 mg to about 330 mg, about 270 mg to about 360 mg, about 270 mg to about 400 mg, about 300 mg to about 330 mg, about 300 mg to about 360 mg, about 300 mg to about 400 mg, about 330 mg to about 360 mg, about 330 mg to about 400 mg, or about 360 mg to about 400 mg. In one embodiment, the specific dose of the cocaine esterase ranges from about 50 mg, about 80 mg, about 110 mg, about 140 mg, about 170 mg, about 210 mg, about 240 mg, about 270 mg, about 300 mg, about 330 mg, about 360 mg, or about 400 mg. In some embodiments, the specific dose of the cocaine esterase ranges from at least about 50 mg, about 80 mg, about 110 mg, about 140 mg, about 170 mg, about 210 mg, about 240 mg, about 270 mg, about 300 mg, about 330 mg, or about 360 mg. In one embodiment, the specific dose of the cocaine esterase ranges from at most about 80 mg, about 110 mg, about 140 mg, about 170 mg, about 210 mg, about 240 mg, about 270 mg, about 300 mg, about 330 mg, about 360 mg, or about 400 mg. In one embodiment, the desired dose of the cocaine esterase is 200 mg.

In one embodiment, the specific dose of the PEGylated cocaine esterase ranges from about 50 mg to about 400 mg. In some embodiments, the specific dose of the PEGylated cocaine esterase ranges from about 50 mg to about 80 mg, about 50 mg to about 110 mg, about 50 mg to about 140 mg, about 50 mg to about 170 mg, about 50 mg to about 210 mg, about 50 mg to about 240 mg, about 50 mg to about 270 mg, about 50 mg to about 300 mg, about 50 mg to about 330 mg, about 50 mg to about 360 mg, about 50 mg to about 400 mg, about 80 mg to about 110 mg, about 80 mg to about 140 mg, about 80 mg to about 170 mg, about 80 mg to about 210 mg, about 80 mg to about 240 mg, about 80 mg to about 270 mg, about 80 mg to about 300 mg, about 80 mg to about 330 mg, about 80 mg to about 360 mg, about 80 mg to about 400 mg, about 110 mg to about 140 mg, about 110 mg to about 170 mg, about 110 mg to about 210 mg, about 110 mg to about 240 mg, about 110 mg to about 270 mg, about 110 mg to about 300 mg, about 110 mg to about 330 mg, about 110 mg to about 360 mg, about 110 mg to about 400 mg, about 140 mg to about 170 mg, about 140 mg to about 210 mg, about 140 mg to about 240 mg, about 140 mg to about 270 mg, about 140 mg to about 300 mg, about 140 mg to about 330 mg, about 140 mg to about 360 mg, about 140 mg to about 400 mg, about 170 mg to about 210 mg, about 170 mg to about 240 mg, about 170 mg to about 270 mg, about 170 mg to about 300 mg, about 170 mg to about 330 mg, about 170 mg to about 360 mg, about 170 mg to about 400 mg, about 210 mg to about 240 mg, about 210 mg to about 270 mg, about 210 mg to about 300 mg, about 210 mg to about 330 mg, about 210 mg to about 360 mg, about 210 mg to about 400 mg, about 240 mg to about 270 mg, about 240 mg to about 300 mg, about 240 mg to about 330 mg, about 240 mg to about 360 mg, about 240 mg to about 400 mg, about 270 mg to about 300 mg, about 270 mg to about 330 mg, about 270 mg to about 360 mg, about 270 mg to about 400 mg, about 300 mg to about 330 mg, about 300 mg to about 360 mg, about 300 mg to about 400 mg, about 330 mg to about 360 mg, about 330 mg to about 400 mg, or about 360 mg to about 400 mg. In one embodiment, the specific dose of the PEGylated cocaine esterase ranges from about 50 mg, about 80 mg, about 110 mg, about 140 mg, about 170 mg, about 210 mg, about 240 mg, about 270 mg, about 300 mg, about 330 mg, about 360 mg, or about 400 mg. In some embodiments, the specific dose of the PEGylated cocaine esterase ranges from at least about 50 mg, about 80 mg, about 110 mg, about 140 mg, about 170 mg, about 210 mg, about 240 mg, about 270 mg, about 300 mg, about 330 mg, or about 360 mg. In one embodiment, the specific dose of the PEGylated cocaine esterase ranges from at most about 80 mg, about 110 mg, about 140 mg, about 170 mg, about 210 mg, about 240 mg, about 270 mg, about 300 mg, about 330 mg, about 360 mg, or about 400 mg. In one embodiment, the desired dose of the PEGylated cocaine esterase ranges from 200 mg.

The oxime compound may assist the cocaine esterase in completing the catalytic cycle, modestly increasing the rate of cocaine esterase-medicated hydrolysis. Thus, the compositions comprising oxime compounds may exhibit faster therapeutic effects (e.g., detoxifying an organophosphate-based agent) than compositions in the absence of oxime compounds. In one embodiment.

The compositions disclosed herein can comprise an oxime compound in a specific amount. In some embodiments, the specific amount of oxime compound is accurate to one significant figure. In another embodiment, the specific amount of oxime compound is accurate to two, three or four significant figures. The presence of the oxime compound in a specific amount in the composition allows for the same desired specific amount of the cocaine esterase to be present in various batches of the composition.

In one embodiment, the specific amount of the total oxime compound content in the composition ranges about 0.1 mg/kg to about 160 mg/kg. In some embodiments, the specific amount of the total oxime compound content in the composition ranges about 0.1 mg/kg to about 0.2 mg/kg, about 0.1 mg/kg to about 0.3 mg/kg, about 0.1 mg/kg to about 0.4 mg/kg, about 0.1 mg/kg to about 0.5 mg/kg, about 0.1 mg/kg to about 0.6 mg/kg, about 0.1 mg/kg to about 126 mg/kg, about 0.1 mg/kg to about 130 mg/kg, about 0.1 mg/kg to about 140 mg/kg, about 0.1 mg/kg to about 150 mg/kg, about 0.1 mg/kg to about 160 mg/kg, about 0.2 mg/kg to about 0.3 mg/kg, about 0.2 mg/kg to about 0.4 mg/kg, about 0.2 mg/kg to about 0.5 mg/kg, about 0.2 mg/kg to about 0.6 mg/kg, about 0.2 mg/kg to about 126 mg/kg, about 0.2 mg/kg to about 130 mg/kg, about 0.2 mg/kg to about 140 mg/kg, about 0.2 mg/kg to about 150 mg/kg, about 0.2 mg/kg to about 160 mg/kg, about 0.3 mg/kg to about 0.4 mg/kg, about 0.3 mg/kg to about 0.5 mg/kg, about 0.3 mg/kg to about 0.6 mg/kg, about 0.3 mg/kg to about 126 mg/kg, about 0.3 mg/kg to about 130 mg/kg, about 0.3 mg/kg to about 140 mg/kg, about 0.3 mg/kg to about 150 mg/kg, about 0.3 mg/kg to about 160 mg/kg, about 0.4 mg/kg to about 0.5 mg/kg, about 0.4 mg/kg to about 0.6 mg/kg, about 0.4 mg/kg to about 126 mg/kg, about 0.4 mg/kg to about 130 mg/kg, about 0.4 mg/kg to about 140 mg/kg, about 0.4 mg/kg to about 150 mg/kg, about 0.4 mg/kg to about 160 mg/kg, about 0.5 mg/kg to about 0.6 mg/kg, about 0.5 mg/kg to about 126 mg/kg, about 0.5 mg/kg to about 130 mg/kg, about 0.5 mg/kg to about 140 mg/kg, about 0.5 mg/kg to about 150 mg/kg, about 0.5 mg/kg to about 160 mg/kg, about 0.6 mg/kg to about 126 mg/kg, about 0.6 mg/kg to about 130 mg/kg, about 0.6 mg/kg to about 140 mg/kg, about 0.6 mg/kg to about 150 mg/kg, about 0.6 mg/kg to about 160 mg/kg, about 126 mg/kg to about 130 mg/kg, about 126 mg/kg to about 140 mg/kg, about 126 mg/kg to about 150 mg/kg, about 126 mg/kg to about 160 mg/kg, about 130 mg/kg to about 140 mg/kg, about 130 mg/kg to about 150 mg/kg, about 130 mg/kg to about 160 mg/kg, about 140 mg/kg to about 150 mg/kg, about 140 mg/kg to about 160 mg/kg, or about 150 mg/kg to about 160 mg/kg. In one embodiment, the specific amount of the total oxime compound content in the composition ranges about 0.1 mg/kg, about 0.2 mg/kg, about 0.3 mg/kg, about 0.4 mg/kg, about 0.5 mg/kg, about 0.6 mg/kg, about 126 mg/kg, about 130 mg/kg, about 140 mg/kg, about 150 mg/kg, or about 160 mg/kg. In one embodiment, the specific amount of the total oxime compound content in the composition ranges at least about 0.1 mg/kg, about 0.2 mg/kg, about 0.3 mg/kg, about 0.4 mg/kg, about 0.5 mg/kg, about 0.6 mg/kg, about 126 mg/kg, about 130 mg/kg, about 140 mg/kg, or about 150 mg/kg. In one embodiment, the specific amount of the total oxime compound content in the composition ranges at most about 0.2 mg/kg, about 0.3 mg/kg, about 0.4 mg/kg, about 0.5 mg/kg, about 0.6 mg/kg, about 126 mg/kg, about 130 mg/kg, about 140 mg/kg, about 150 mg/kg, or about 160 mg/kg. In yet another exemplary embodiment, the desired amount of the total oxime compound ranges in the from 0.6 to 126 mg/kg of the composition.

In one embodiment, the specific amount of the oxime compound in the composition, by weight, ranges from about 0.1% to about 6%. In some embodiments, the specific amount of the oxime compound in the composition, by weight, ranges from about 0.1% to about 0.5%, about 0.1% to about 1%, about 0.1% to about 2%, about 0.1% to about 2.5%, about 0.1% to about 3%, about 0.1% to about 3.5%, about 0.1% to about 4%, about 0.1% to about 4.5%, about 0.1% to about 4%, about 0.1% to about 5.5%, about 0.1% to about 6%, about 0.5% to about 1%, about 0.5% to about 2%, about 0.5% to about 2.5%, about 0.5% to about 3%, about 0.5% to about 3.5%, about 0.5% to about 4%, about 0.5% to about 4.5%, about 0.5% to about 4%, about 0.5% to about 5.5%, about 0.5% to about 6%, about 1% to about 2%, about 1% to about 2.5%, about 1% to about 3%, about 1% to about 3.5%, about 1% to about 4%, about 1% to about 4.5%, about 1% to about 4%, about 1% to about 5.5%, about 1% to about 6%, about 2% to about 2.5%, about 2% to about 3%, about 2% to about 3.5%, about 2% to about 4%, about 2% to about 4.5%, about 2% to about 4%, about 2% to about 5.5%, about 2% to about 6%, about 2.5% to about 3%, about 2.5% to about 3.5%, about 2.5% to about 4%, about 2.5% to about 4.5%, about 2.5% to about 4%, about 2.5% to about 5.5%, about 2.5% to about 6%, about 3% to about 3.5%, about 3% to about 4%, about 3% to about 4.5%, about 3% to about 4%, about 3% to about 5.5%, about 3% to about 6%, about 3.5% to about 4%, about 3.5% to about 4.5%, about 3.5% to about 4%, about 3.5% to about 5.5%, about 3.5% to about 6%, about 4% to about 4.5%, about 4% to about 4%, about 4% to about 5.5%, about 4% to about 6%, about 4.5% to about 4%, about 4.5% to about 5.5%, about 4.5% to about 6%, about 4% to about 5.5%, about 4% to about 6%, or about 5.5% to about 6%. In one embodiment, the specific amount of the oxime compound in the composition, by weight, ranges from about 0.1%, about 0.5%, about 1%, about 2%, about 2.5%, about 3%, about 3.5%, about 4%, about 4.5%, about 4%, about 5.5%, or about 6%. In one embodiment, the specific amount of the oxime compound in the composition, by weight, ranges from at least about 0.1%, about 0.5%, about 1%, about 2%, about 2.5%, about 3%, about 3.5%, about 4%, about 4.5%, about 4%, or about 5.5%. In one embodiment, the specific amount of the oxime compound in the composition, by weight, ranges from at most about 0.5%, about 1%, about 2%, about 2.5%, about 3%, about 3.5%, about 4%, about 4.5%, about 4%, about 5.5%, or about 6%. In one embodiment, the desired amount of the oxime compound in the composition, by weight, is about 3%.

The pharmaceutically acceptable excipient may stabilize the cocaine esterase in the composition, modestly increasing the rate of cocaine esterase-medicated hydrolysis or shelf-life period of cocaine esterase. The pharmaceutically acceptable excipient may also stabilize the cocaine esterase and the oxime compound in the composition, modestly increasing the rate of cocaine esterase-medicated hydrolysis or shelf-life period of cocaine esterase. Thus, the compositions comprising pharmaceutically acceptable excipient may exhibit faster therapeutic effects (e.g., detoxifying an organophosphate-based agent) than compositions in the absence of pharmaceutically acceptable excipient.

The composition disclosed herein can comprise one or more pharmaceutically acceptable excipients in a specific amount. In some embodiments, the specific amount of one or more pharmaceutically acceptable excipients is accurate to one significant figure. In another embodiment, the specific amount of one or more pharmaceutically acceptable excipients is accurate to two, three or four significant figures. The presence of the one or more pharmaceutically acceptable excipients in a specific amount in the composition allows for the same desired specific amount of the cocaine esterase to be present in various batches of the composition.

In some embodiments, a cocaine esterase and an oxime compound are present in a proportion such that the specific ratio of cocaine esterase to oxime compound ranges from about 0.1:1 to about 1:0.1. In some embodiments, a cocaine esterase and an oxime compound are present in a proportion such that the specific ratio of cocaine esterase to oxime compound ranges from about 0.3:1 to about 1:0.1. In some embodiments, a cocaine esterase and an oxime compound are present in a proportion such that the specific ratio of cocaine esterase to oxime compound ranges from about 0.5:1 to about 1:0.1. In some embodiments, a cocaine esterase and an oxime compound are present in a proportion such that the specific ratio of cocaine esterase to oxime compound ranges from about 0.7:1 to about 1:0.1. In some embodiments, a cocaine esterase and an oxime compound are present in a proportion such that the specific ratio of cocaine esterase to oxime compound ranges from about 0.9:1 to about 1:0.1. In some embodiments, a cocaine esterase and an oxime compound are present in a proportion such that the specific ratio of cocaine esterase to oxime compound ranges from about 0.1:1 to about 1:0.3. In some embodiments, a cocaine esterase and an oxime compound are present in a proportion such that the specific ratio of cocaine esterase to oxime compound ranges from about 0.1:1 to about 1:0.5. In some embodiments, a cocaine esterase and an oxime compound are present in a proportion such that the specific ratio of cocaine esterase to oxime compound ranges from about 0.1:1 to about 1:0.7. In some embodiments, a cocaine esterase and an oxime compound are present in a proportion such that the specific ratio of cocaine esterase to oxime compound ranges from about 0.1:1 to about 1:0.9. In some embodiments, a cocaine esterase and an oxime compound are present in a proportion such that the desired ratio of cocaine esterase to oxime compound ranges from about 0.1:1 to about 1:0.1.

In some embodiments, the recombinant CocE composition has a specific CocE concentration, by weight of the composition, ranging about 1 mg/mL to about 200 mg/mL. In some embodiments, the recombinant CocE compositions disclosed herein have a specific CocE concentration, by weight of the composition, ranging about 1 mg/mL to about 4 mg/mL, about 1 mg/mL to about 5 mg/mL, about 1 mg/mL to about 10 mg/mL, about 1 mg/mL to about 20 mg/mL, about 1 mg/mL to about 30 mg/mL, about 1 mg/mL to about 50 mg/mL, about 1 mg/mL to about 60 mg/mL, about 1 mg/mL to about 80 mg/mL, about 1 mg/mL to about 100 mg/mL, about 1 mg/mL to about 150 mg/mL, about 1 mg/mL to about 200 mg/mL, about 4 mg/mL to about 5 mg/mL, about 4 mg/mL to about 10 mg/mL, about 4 mg/mL to about 20 mg/mL, about 4 mg/mL to about 30 mg/mL, about 4 mg/mL to about 50 mg/mL, about 4 mg/mL to about 60 mg/mL, about 4 mg/mL to about 80 mg/mL, about 4 mg/mL to about 100 mg/mL, about 4 mg/mL to about 150 mg/mL, about 4 mg/mL to about 200 mg/mL, about 5 mg/mL to about 10 mg/mL, about 5 mg/mL to about 20 mg/mL, about 5 mg/mL to about 30 mg/mL, about 5 mg/mL to about 50 mg/mL, about 5 mg/mL to about 60 mg/mL, about 5 mg/mL to about 80 mg/mL, about 5 mg/mL to about 100 mg/mL, about 5 mg/mL to about 150 mg/mL, about 5 mg/mL to about 200 mg/mL, about 10 mg/mL to about 20 mg/mL, about 10 mg/mL to about 30 mg/mL, about 10 mg/mL to about 50 mg/mL, about 10 mg/mL to about 60 mg/mL, about 10 mg/mL to about 80 mg/mL, about 10 mg/mL to about 100 mg/mL, about 10 mg/mL to about 150 mg/mL, about 10 mg/mL to about 200 mg/mL, about 20 mg/mL to about 30 mg/mL, about 20 mg/mL to about 50 mg/mL, about 20 mg/mL to about 60 mg/mL, about 20 mg/mL to about 80 mg/mL, about 20 mg/mL to about 100 mg/mL, about 20 mg/mL to about 150 mg/mL, about 20 mg/mL to about 200 mg/mL, about 30 mg/mL to about 50 mg/mL, about 30 mg/mL to about 60 mg/mL, about 30 mg/mL to about 80 mg/mL, about 30 mg/mL to about 100 mg/mL, about 30 mg/mL to about 150 mg/mL, about 30 mg/mL to about 200 mg/mL, about 50 mg/mL to about 60 mg/mL, about 50 mg/mL to about 80 mg/mL, about 50 mg/mL to about 100 mg/mL, about 50 mg/mL to about 150 mg/mL, about 50 mg/mL to about 200 mg/mL, about 60 mg/mL to about 80 mg/mL, about 60 mg/mL to about 100 mg/mL, about 60 mg/mL to about 150 mg/mL, about 60 mg/mL to about 200 mg/mL, about 80 mg/mL to about 100 mg/mL, about 80 mg/mL to about 150 mg/mL, about 80 mg/mL to about 200 mg/mL, about 100 mg/mL to about 150 mg/mL, about 100 mg/mL to about 200 mg/mL, or about 150 mg/mL to about 200 mg/mL. In some embodiments, the recombinant CocE composition has the specific CocE concentration, by weight of the composition, ranging about 1 mg/mL, about 4 mg/mL, about 5 mg/mL, about 10 mg/mL, about 20 mg/mL, about 30 mg/mL, about 50 mg/mL, about 60 mg/mL, about 80 mg/mL, about 100 mg/mL, about 150 mg/mL, or about 200 mg/mL. In some embodiments, the recombinant CocE composition has the specific CocE concentration, by weight of the composition, ranging at least about 1 mg/mL, about 4 mg/mL, about 5 mg/mL, about 10 mg/mL, about 20 mg/mL, about 30 mg/mL, about 50 mg/mL, about 60 mg/mL, about 80 mg/mL, about 100 mg/mL, or about 150 mg/mL. In some embodiments, the recombinant CocE composition has the specific CocE concentration, by weight of the composition, ranging at most about 4 mg/mL, about 5 mg/mL, about 10 mg/mL, about 20 mg/mL, about 30 mg/mL, about 50 mg/mL, about 60 mg/mL, about 80 mg/mL, about 100 mg/mL, about 150 mg/mL, or about 200 mg/mL. In some embodiments, the desired recombinant CocE composition has the CocE concentration, by weight of the composition, ranging from 5 to 100 mg/mL. In some embodiments, higher concentration than 100 mg/mL may cause a gelation, unsuitable for administration by injection.

In some embodiments, the recombinant CocE composition has the specific amount of CocE, by weight of the composition, ranging about 0.1% to about 20%. In some embodiments, the recombinant CocE composition has the specific amount of CocE, by weight of the composition, ranging about 0.1% to about 0.2%, about 0.1% to about 0.3%, about 0.1% to about 0.4%, about 0.1% to about 0.5%, about 0.1% to about 1%, about 0.1% to about 2%, about 0.1% to about 8%, about 0.1% to about 9%, about 0.1% to about 10%, about 0.1% to about 15%, about 0.1% to about 20%, about 0.2% to about 0.3%, about 0.2% to about 0.4%, about 0.2% to about 0.5%, about 0.2% to about 1%, about 0.2% to about 2%, about 0.2% to about 8%, about 0.2% to about 9%, about 0.2% to about 10%, about 0.2% to about 15%, about 0.2% to about 20%, about 0.3% to about 0.4%, about 0.3% to about 0.5%, about 0.3% to about 1%, about 0.3% to about 2%, about 0.3% to about 8%, about 0.3% to about 9%, about 0.3% to about 10%, about 0.3% to about 15%, about 0.3% to about 20%, about 0.4% to about 0.5%, about 0.4% to about 1%, about 0.4% to about 2%, about 0.4% to about 8%, about 0.4% to about 9%, about 0.4% to about 10%, about 0.4% to about 15%, about 0.4% to about 20%, about 0.5% to about 1%, about 0.5% to about 2%, about 0.5% to about 8%, about 0.5% to about 9%, about 0.5% to about 10%, about 0.5% to about 15%, about 0.5% to about 20%, about 1% to about 2%, about 1% to about 8%, about 1% to about 9%, about 1% to about 10%, about 1% to about 15%, about 1% to about 20%, about 2% to about 8%, about 2% to about 9%, about 2% to about 10%, about 2% to about 15%, about 2% to about 20%, about 8% to about 9%, about 8% to about 10%, about 8% to about 15%, about 8% to about 20%, about 9% to about 10%, about 9% to about 15%, about 9% to about 20%, about 10% to about 15%, about 10% to about 20%, or about 15% to about 20%. In some embodiments, the recombinant CocE composition has the specific amount of CocE, by weight of the composition, ranging about 0.1%, about 0.2%, about 0.3%, about 0.4%, about 0.5%, about 1%, about 2%, about 8%, about 9%, about 10%, about 15%, or about 20%. In some embodiments, the recombinant CocE composition has the specific amount of CocE, by weight of the composition, ranging at least about 0.1%, about 0.2%, about 0.3%, about 0.4%, about 0.5%, about 1%, about 2%, about 8%, about 9%, about 10%, or about 15%. In some embodiments, the recombinant CocE composition has the specific amount of CocE, by weight of the composition, ranging at most about 0.2%, about 0.3%, about 0.4%, about 0.5%, about 1%, about 2%, about 8%, about 9%, about 10%, about 15%, or about 20%. In some embodiments, the recombinant CocE composition has the desired amount of CocE, by weight of the composition, ranging from 0.5 to 10%.

In some embodiments, the specific recombinant CocE composition has an oxime compound concentration, by weight of the composition, ranging from about 0.1 mg/mL to about 20 mg/mL. In some embodiments, the specific recombinant CocE composition has an oxime compound concentration, by weight of the composition, ranging from about 0.1 mg/mL to about 0.2 mg/mL, about 0.1 mg/mL to about 0.3 mg/mL, about 0.1 mg/mL to about 0.4 mg/mL, about 0.1 mg/mL to about 0.5 mg/mL, about 0.1 mg/mL to about 1 mg/mL, about 0.1 mg/mL to about 2 mg/mL, about 0.1 mg/mL to about 8 mg/mL, about 0.1 mg/mL to about 9 mg/mL, about 0.1 mg/mL to about 10 mg/mL, about 0.1 mg/mL to about 15 mg/mL, about 0.1 mg/mL to about 20 mg/mL, about 0.2 mg/mL to about 0.3 mg/mL, about 0.2 mg/mL to about 0.4 mg/mL, about 0.2 mg/mL to about 0.5 mg/mL, about 0.2 mg/mL to about 1 mg/mL, about 0.2 mg/mL to about 2 mg/mL, about 0.2 mg/mL to about 8 mg/mL, about 0.2 mg/mL to about 9 mg/mL, about 0.2 mg/mL to about 10 mg/mL, about 0.2 mg/mL to about 15 mg/mL, about 0.2 mg/mL to about 20 mg/mL, about 0.3 mg/mL to about 0.4 mg/mL, about 0.3 mg/mL to about 0.5 mg/mL, about 0.3 mg/mL to about 1 mg/mL, about 0.3 mg/mL to about 2 mg/mL, about 0.3 mg/mL to about 8 mg/mL, about 0.3 mg/mL to about 9 mg/mL, about 0.3 mg/mL to about 10 mg/mL, about 0.3 mg/mL to about 15 mg/mL, about 0.3 mg/mL to about 20 mg/mL, about 0.4 mg/mL to about 0.5 mg/mL, about 0.4 mg/mL to about 1 mg/mL, about 0.4 mg/mL to about 2 mg/mL, about 0.4 mg/mL to about 8 mg/mL, about 0.4 mg/mL to about 9 mg/mL, about 0.4 mg/mL to about 10 mg/mL, about 0.4 mg/mL to about 15 mg/mL, about 0.4 mg/mL to about 20 mg/mL, about 0.5 mg/mL to about 1 mg/mL, about 0.5 mg/mL to about 2 mg/mL, about 0.5 mg/mL to about 8 mg/mL, about 0.5 mg/mL to about 9 mg/mL, about 0.5 mg/mL to about 10 mg/mL, about 0.5 mg/mL to about 15 mg/mL, about 0.5 mg/mL to about 20 mg/mL, about 1 mg/mL to about 2 mg/mL, about 1 mg/mL to about 8 mg/mL, about 1 mg/mL to about 9 mg/mL, about 1 mg/ml to about 10 mg/mL, about 1 mg/mL to about 15 mg/mL, about 1 mg/mL to about 20 mg/mL, about 2 mg/mL to about 8 mg/mL, about 2 mg/mL to about 9 mg/mL, about 2 mg/mL to about 10 mg/mL, about 2 mg/mL to about 15 mg/mL, about 2 mg/mL to about 20 mg/mL, about 8 mg/mL to about 9 mg/mL, about 8 mg/mL to about 10 mg/mL, about 8 mg/mL to about 15 mg/mL, about 8 mg/mL to about 20 mg/mL, about 9 mg/mL to about 10 mg/mL, about 9 mg/mL to about 15 mg/mL, about 9 mg/mL to about 20 mg/mL, about 10 mg/mL to about 15 mg/mL, about 10 mg/mL to about 20 mg/mL, or about 15 mg/mL to about 20 mg/mL. In some embodiments, the specific recombinant CocE composition has an oxime compound concentration, by weight of the composition, ranging from about 0.1 mg/mL, about 0.2 mg/mL, about 0.3 mg/mL, about 0.4 mg/mL, about 0.5 mg/mL, about 1 mg/mL, about 2 mg/mL, about 8 mg/mL, about 9 mg/mL, about 10 mg/mL, about 15 mg/mL, or about 20 mg/mL. In some embodiments, the specific recombinant CocE composition has an oxime compound concentration, by weight of the composition, ranging from at least about 0.1 mg/mL, about 0.2 mg/mL, about 0.3 mg/mL, about 0.4 mg/mL, about 0.5 mg/mL, about 1 mg/mL, about 2 mg/mL, about 8 mg/mL, about 9 mg/mL, about 10 mg/mL, or about 15 mg/mL. In some embodiments, the specific recombinant CocE composition has an oxime compound concentration, by weight of the composition, ranging from at most about 0.2 mg/mL, about 0.3 mg/mL, about 0.4 mg/mL, about 0.5 mg/mL, about 1 mg/mL, about 2 mg/mL, about 8 mg/mL, about 9 mg/mL, about 10 mg/mL, about 15 mg/mL, or about 20 mg/mL. In some embodiments, the desired recombinant CocE composition has an oxime compound concentration, by weight of the composition, ranging from 0.5 to 10 mg/mL.

In some embodiments, the recombinant CocE composition has a specific amount of oxime compound, by weight of the composition, ranging from about 0.01% to about 5%. In some embodiments, the recombinant CocE composition has a specific amount of oxime compound, by weight of the composition, ranging from about 0.01% to about 0.02%, about 0.01% to about 0.03%, about 0.01% to about 0.04%, about 0.01% to about 0.05%, about 0.01% to about 1%, about 0.01% to about 2%, about 0.01% to about 1%, about 0.01% to about 2%, about 0.01% to about 3%, about 0.01% to about 4%, about 0.01% to about 5%, about 0.02% to about 0.03%, about 0.02% to about 0.04%, about 0.02% to about 0.05%, about 0.02% to about 1%, about 0.02% to about 2%, about 0.02% to about 1%, about 0.02% to about 2%, about 0.02% to about 3%, about 0.02% to about 4%, about 0.02% to about 5%, about 0.03% to about 0.04%, about 0.03% to about 0.05%, about 0.03% to about 1%, about 0.03% to about 2%, about 0.03% to about 1%, about 0.03% to about 2%, about 0.03% to about 3%, about 0.03% to about 4%, about 0.03% to about 5%, about 0.04% to about 0.05%, about 0.04% to about 1%, about 0.04% to about 2%, about 0.04% to about 1%, about 0.04% to about 2%, about 0.04% to about 3%, about 0.04% to about 4%, about 0.04% to about 5%, about 0.05% to about 1%, about 0.05% to about 2%, about 0.05% to about 1%, about 0.05% to about 2%, about 0.05% to about 3%, about 0.05% to about 4%, about 0.05% to about 5%, about 1% to about 2%, about 1% to about 1%, about 1% to about 2%, about 1% to about 3%, about 1% to about 4%, about 1% to about 5%, about 2% to about 1%, about 2% to about 2%, about 2% to about 3%, about 2% to about 4%, about 2% to about 5%, about 1% to about 2%, about 1% to about 3%, about 1% to about 4%, about 1% to about 5%, about 2% to about 3%, about 2% to about 4%, about 2% to about 5%, about 3% to about 4%, about 3% to about 5%, or about 4% to about 5%. In some embodiments, the recombinant CocE composition has a specific amount of oxime compound, by weight of the composition, ranging from about 0.01%, about 0.02%, about 0.03%, about 0.04%, about 0.05%, about 1%, about 2%, about 1%, about 2%, about 3%, about 4%, or about 5%. In some embodiments, the recombinant CocE composition has a specific amount of oxime compound, by weight of the composition, ranging from at least about 0.01%, about 0.02%, about 0.03%, about 0.04%, about 0.05%, about 1%, about 2%, about 1%, about 2%, about 3%, or about 4%. In some embodiments, the recombinant CocE composition has a specific amount of oxime compound, by weight of the composition, ranging from at most about 0.02%, about 0.03%, about 0.04%, about 0.05%, about 1%, about 2%, about 1%, about 2%, about 3%, about 4%, or about 5%. In some embodiments, the recombinant CocE composition has a desired amount of oxime compound, by weight of the composition, ranging from 0.05 to 1%.

In some embodiments, the recombinant CocE composition has a total amount of CocE and oxime compound, by weight of the composition, ranging from about 0.1% to about 20%. In some embodiments, the recombinant CocE composition has a total amount of CocE and oxime compound, by weight of the composition, ranging from about 0.1% to about 0.2%, about 0.1% to about 0.3%, about 0.1% to about 0.4%, about 0.1% to about 0.5%, about 0.1% to about 0.55%, about 0.1% to about 11%, about 0.1% to about 12%, about 0.1% to about 13%, about 0.1% to about 14%, about 0.1% to about 15%, about 0.1% to about 20%, about 0.2% to about 0.3%, about 0.2% to about 0.4%, about 0.2% to about 0.5%, about 0.2% to about 0.55%, about 0.2% to about 11%, about 0.2% to about 12%, about 0.2% to about 13%, about 0.2% to about 14%, about 0.2% to about 15%, about 0.2% to about 20%, about 0.3% to about 0.4%, about 0.3% to about 0.5%, about 0.3% to about 0.55%, about 0.3% to about 11%, about 0.3% to about 12%, about 0.3% to about 13%, about 0.3% to about 14%, about 0.3% to about 15%, about 0.3% to about 20%, about 0.4% to about 0.5%, about 0.4% to about 0.55%, about 0.4% to about 11%, about 0.4% to about 12%, about 0.4% to about 13%, about 0.4% to about 14%, about 0.4% to about 15%, about 0.4% to about 20%, about 0.5% to about 0.55%, about 0.5% to about 11%, about 0.5% to about 12%, about 0.5% to about 13%, about 0.5% to about 14%, about 0.5% to about 15%, about 0.5% to about 20%, about 0.55% to about 11%, about 0.55% to about 12%, about 0.55% to about 13%, about 0.55% to about 14%, about 0.55% to about 15%, about 0.55% to about 20%, about 11% to about 12%, about 11% to about 13%, about 11% to about 14%, about 11% to about 15%, about 11% to about 20%, about 12% to about 13%, about 12% to about 14%, about 12% to about 15%, about 12% to about 20%, about 13% to about 14%, about 13% to about 15%, about 13% to about 20%, about 14% to about 15%, about 14% to about 20%, or about 15% to about 20%. In some embodiments, the recombinant CocE composition has a total amount of CocE and oxime compound, by weight of the composition, ranging from about 0.1%, about 0.2%, about 0.3%, about 0.4%, about 0.5%, about 0.55%, about 11%, about 12%, about 13%, about 14%, about 15%, or about 20%. In some embodiments, the recombinant CocE composition has a total amount of CocE and oxime compound, by weight of the composition, ranging from at least about 0.1%, about 0.2%, about 0.3%, about 0.4%, about 0.5%, about 0.55%, about 11%, about 12%, about 13%, about 14%, or about 15%. In some embodiments, the recombinant CocE composition has a total amount of CocE and oxime compound, by weight of the composition, ranging from at most about 0.2%, about 0.3%, about 0.4%, about 0.5%, about 0.55%, about 11%, about 12%, about 13%, about 14%, about 15%, or about 20%. In some embodiments, the desired recombinant CocE composition has a total concentration of CocE and oxime compound, by weight of the composition, ranging from 0.55 to 11%.

In some embodiments, the recombinant CocE is active for greater than or equal to 1 h at 37° C. In some embodiments, the recombinant CocE is active for greater than or equal to 2 h at 37° C. In some embodiments, the recombinant CocE is active for greater than or equal to 3 h at 37° C. In some embodiments, the recombinant CocE is active for greater than or equal to 4 h at 37° C. In some embodiments, the recombinant CocE is active for greater than or equal to 5 h at 37° C. In some embodiments, the recombinant CocE is active for greater than or equal to 6 h at 37° C.

In some embodiments, maximum initial velocity of a reaction (Vₘₐₓ) of the recombinant CocE in the composition ranges from 1200 μmol/min to 12,000 μmol/min.

The compositions disclosed herein can be present in a powder form. The components of the composition can also be in powder form. The compositions disclosed herein may be in the form of a free-flowing powder depending on the embodiment. Such compositions are thus easy to handle during manufacturing and packaging processes. Further, the dry, free-flowing powder form allows the composition to be free from clumps and not be as susceptible to microbial growth as a composition with clumping due to moisture absorption.

In some embodiments, the compositions disclosed herein are in a liquid form. The components of the composition can also be in a liquid form. The compositions disclosed herein may be in the form of a free-flowing liquid depending on the embodiment. Such compositions are thus easy to handle during manufacturing and packaging processes. Further, the free-flowing liquid form allows the composition to be free from aggregation.

In some embodiments, upon contacting the cocaine esterase with the organophosphate-based agent, an amount of the organophosphate-based agent is decreased to no more than about 25% of the original amount after no more than about 10 hours, no more than about 9 hours, no more than about 8 hours, no more than about 7 hours, no more than about 6 hours, no more than about 5 hours, no more than about 4 hours, no more than about 3 hours, no more than about 2 hours, no more than about 1 hour, no more than about 50 minutes, no more than about 45 minutes, no more than about 40 minutes, no more than about 35 minutes, no more than about 30 minutes, no more than about 25 minutes, no more than about 20 minutes, no more than about 15 minutes, no more than about 10 minutes, no more than about 9 minutes, no more than about 8 minutes, no more than about 7 minutes, no more than about 6 minutes, no more than about 5 minutes, no more than about 4 minutes, no more than about 3 minutes, no more than about 2 minutes, no more than about 1 minute (60 seconds), no more than about 55 seconds, no more than about 50 seconds, no more than about 45 seconds, no more than about 40 seconds, no more than about 35 seconds, no more than about 30 seconds, no more than about 25 seconds, no more than about 20 seconds, no more than about 15 seconds, no more than about 10 seconds, or no more than about 5 seconds.

In some embodiments, upon contacting the cocaine esterase with the organophosphate-based agent, an amount of the organophosphate-based agent is decreased to no more than about 50%, no more than about 45%, no more than about 40%, no more than about 35%, no more than about 30%, no more than 25%, no more than about 20%, no more than about 15%, no more than about 10%, no more than about 5%, no more than about 4%, no more than about 3%, no more than about 2% or no more than about 1% of the original amount after no more than about 10 hours, no more than about 9 hours, no more than about 8 hours, no more than about 7 hours, no more than about 6 hours, no more than about 5 hours, no more than about 4 hours, no more than about 3 hours, no more than about 2 hours, no more than about 1 hour, no more than about 50 minutes, no more than about 45 minutes, no more than about 40 minutes, no more than about 35 minutes, no more than about 30 minutes, no more than about 25 minutes, no more than about 20 minutes, no more than about 15 minutes, no more than about 10 minutes, no more than about 9 minutes, no more than about 8 minutes, no more than about 7 minutes, no more than about 6 minutes, no more than about 5 minutes, no more than about 4 minutes, no more than about 3 minutes, no more than about 2 minutes, no more than about 1 minute (60 seconds), no more than about 55 seconds, no more than about 50 seconds, no more than about 45 seconds, no more than about 40 seconds, no more than about 35 seconds, no more than about 30 seconds, no more than about 25 seconds, no more than about 20 seconds, no more than about 15 seconds, no more than about 10 seconds, or no more than about 5 seconds.

In some embodiments, upon contacting the cocaine esterase with the organophosphate-based agent, an amount of the organophosphate-based agent is decreased to no more than about 25% of the original amount after no more than about 60 minutes. In some embodiments, upon contacting the cocaine esterase with the organophosphate-based agent, an amount of the organophosphate-based agent is decreased to no more than about 25% of the original amount after no more than about 50 minutes. In some embodiments, upon contacting the cocaine esterase with the organophosphate-based agent, an amount of the organophosphate-based agent is decreased to no more than about 25% of the original amount after no more than about 40 minutes. In some embodiments, upon contacting the cocaine esterase with the organophosphate-based agent, an amount of the organophosphate-based agent is decreased to no more than about 25% of the original amount after no more than about 30 minutes. In some embodiments, upon contacting the cocaine esterase with the organophosphate-based agent, an amount of the organophosphate-based agent is decreased to no more than about 25% of the original amount after no more than about 20 minutes. In some embodiments, upon contacting the cocaine esterase with the organophosphate-based agent, an amount of the organophosphate-based agent is decreased to no more than about 25% of the original amount after no more than about 10 minutes. In some embodiments, upon contacting the cocaine esterase with the organophosphate-based agent, an amount of the organophosphate-based agent is decreased to no more than about 25% of the original amount after no more than about 9 minutes. In some embodiments, upon contacting the cocaine esterase with the organophosphate-based agent, an amount of the organophosphate-based agent is decreased to no more than about 25% of the original amount after no more than about 8 minutes. In some embodiments, upon contacting the cocaine esterase with the organophosphate-based agent, an amount of the organophosphate-based agent is decreased to no more than about 25% of the original amount after no more than about 7 minutes. In some embodiments, upon contacting the cocaine esterase with the organophosphate-based agent, an amount of the organophosphate-based agent is decreased to no more than about 25% of the original amount after no more than about 6 minutes. In some embodiments, upon contacting the cocaine esterase with the organophosphate-based agent, an amount of the organophosphate-based agent is decreased to no more than about 25% of the original amount after no more than about 5 minutes. In some embodiments, upon contacting the cocaine esterase with the organophosphate-based agent, an amount of the organophosphate-based agent is decreased to no more than about 25% of the original amount after no more than about 4 minutes. In some embodiments, upon contacting the cocaine esterase with the organophosphate-based agent, an amount of the organophosphate-based agent is decreased to no more than about 25% of the original amount after no more than about 3 minutes. In some embodiments, upon contacting the cocaine esterase with the organophosphate-based agent, an amount of the organophosphate-based agent is decreased to no more than about 25% of the original amount after no more than about 2 minutes. In some embodiments, upon contacting the cocaine esterase with the organophosphate-based agent, an amount of the organophosphate-based agent is decreased to no more than about 25% of the original amount after no more than about 1 minute (or 60 seconds). In some embodiments, upon contacting the cocaine esterase with the organophosphate-based agent, an amount of the organophosphate-based agent is decreased to no more than about 25% of the original amount after no more than about 50 seconds. In some embodiments, upon contacting the cocaine esterase with the organophosphate-based agent, an amount of the organophosphate-based agent is decreased to no more than about 25% of the original amount after no more than about 40 seconds. In some embodiments, upon contacting the cocaine esterase with the organophosphate-based agent, an amount of the organophosphate-based agent is decreased to no more than about 25% of the original amount after no more than about 30 seconds. In some embodiments, upon contacting the cocaine esterase with the organophosphate-based agent, an amount of the organophosphate-based agent is decreased to no more than about 25% of the original amount after no more than about 20 seconds. In some embodiments, upon contacting the cocaine esterase with the organophosphate-based agent, an amount of the organophosphate-based agent is decreased to no more than about 25% of the original amount after no more than about 10 seconds.

In some embodiments, upon contacting the cocaine esterase with the organophosphate-based agent, an amount of the organophosphate-based agent is decreased to no more than about 75% of the original amount after no more than about 30 minutes. In some embodiments, upon contacting the cocaine esterase with the organophosphate-based agent, an amount of the organophosphate-based agent is decreased to no more than about 70% of the original amount after no more than about 30 minutes. In some embodiments, upon contacting the cocaine esterase with the organophosphate-based agent, an amount of the organophosphate-based agent is decreased to no more than about 65% of the original amount after no more than about 30 minutes. In some embodiments, upon contacting the cocaine esterase with the organophosphate-based agent, an amount of the organophosphate-based agent is decreased to no more than about 60% of the original amount after no more than about 30 minutes. In some embodiments, upon contacting the cocaine esterase with the organophosphate-based agent, an amount of the organophosphate-based agent is decreased to no more than about 55% of the original amount after no more than about 30 minutes. In some embodiments, upon contacting the cocaine esterase with the organophosphate-based agent, an amount of the organophosphate-based agent is decreased to no more than about 50% of the original amount after no more than about 30 minutes. In some embodiments, upon contacting the cocaine esterase with the organophosphate-based agent, an amount of the organophosphate-based agent is decreased to no more than about 45% of the original amount after no more than about 30 minutes. In some embodiments, upon contacting the cocaine esterase with the organophosphate-based agent, an amount of the organophosphate-based agent is decreased to no more than about 40% of the original amount after no more than about 30 minutes. In some embodiments, upon contacting the cocaine esterase with the organophosphate-based agent, an amount of the organophosphate-based agent is decreased to no more than about 35% of the original amount after no more than about 30 minutes. In some embodiments, upon contacting the cocaine esterase with the organophosphate-based agent, an amount of the organophosphate-based agent is decreased to no more than about 30% of the original amount after no more than about 30 minutes. In some embodiments, upon contacting the cocaine esterase with the organophosphate-based agent, an amount of the organophosphate-based agent is decreased to no more than about 25% of the original amount after no more than about 30 minutes. In some embodiments, upon contacting the cocaine esterase with the organophosphate-based agent, an amount of the organophosphate-based agent is decreased to no more than about 20% of the original amount after no more than about 30 minutes. In some embodiments, upon contacting the cocaine esterase with the organophosphate-based agent, an amount of the organophosphate-based agent is decreased to no more than about 15% of the original amount after no more than about 30 minutes. In some embodiments, upon contacting the cocaine esterase with the organophosphate-based agent, an amount of the organophosphate-based agent is decreased to no more than about 10% of the original amount after no more than about 30 minutes. In some embodiments, upon contacting the cocaine esterase with the organophosphate-based agent, an amount of the organophosphate-based agent is decreased to no more than about 5% of the original amount after no more than about 30 minutes.

In some embodiments, upon contacting the cocaine esterase with the organophosphate-based agent, an amount of the organophosphate-based agent ranges from about 75% to about 1% of the original amount after no more than about 30 minutes. In some embodiments, upon contacting the cocaine esterase with the organophosphate-based agent, an amount of the organophosphate-based agent ranges from about 70% to about 1% of the original amount after no more than about 30 minutes. In some embodiments, upon contacting the cocaine esterase with the organophosphate-based agent, an amount of the organophosphate-based agent ranges from about 65% to about 1% of the original amount after no more than about 30 minutes. In some embodiments, upon contacting the cocaine esterase with the organophosphate-based agent, an amount of the organophosphate-based agent ranges from about 60% to about 1% of the original amount after no more than about 30 minutes. In some embodiments, upon contacting the cocaine esterase with the organophosphate-based agent, an amount of the organophosphate-based agent ranges from about 55% to about 1% of the original amount after no more than about 30 minutes. In some embodiments, upon contacting the cocaine esterase with the organophosphate-based agent, an amount of the organophosphate-based agent ranges from about 50% to about 1% of the original amount after no more than about 30 minutes. In some embodiments, upon contacting the cocaine esterase with the organophosphate-based agent, an amount of the organophosphate-based agent ranges from about 45% to about 1% of the original amount after no more than about 30 minutes. In some embodiments, upon contacting the cocaine esterase with the organophosphate-based agent, an amount of the organophosphate-based agent ranges from about 40% to about 1% of the original amount after no more than about 30 minutes. In some embodiments, upon contacting the cocaine esterase with the organophosphate-based agent, an amount of the organophosphate-based agent ranges from about 35% to about 1% of the original amount after no more than about 30 minutes. In some embodiments, upon contacting the cocaine esterase with the organophosphate-based agent, an amount of the organophosphate-based agent ranges from about 30% to about 1% of the original amount after no more than about 30 minutes. In some embodiments, upon contacting the cocaine esterase with the organophosphate-based agent, an amount of the organophosphate-based agent ranges from about 25% to about 1% of the original amount after no more than about 30 minutes. In some embodiments, upon contacting the cocaine esterase with the organophosphate-based agent, an amount of the organophosphate-based agent ranges from about 20% to about 1% of the original amount after no more than about 30 minutes. In some embodiments, upon contacting the cocaine esterase with the organophosphate-based agent, an amount of the organophosphate-based agent ranges from about 15% to about 1% of the original amount after no more than about 30 minutes. In some embodiments, upon contacting the cocaine esterase with the organophosphate-based agent, an amount of the organophosphate-based agent ranges from about 10% to about 1% of the original amount after no more than about 30 minutes. In some embodiments, upon contacting the cocaine esterase with the organophosphate-based agent, an amount of the organophosphate-based agent ranges from about 5% to about 1% of the original amount after no more than about 30 minutes.

### Methods of Manufacturing a CocE Composition

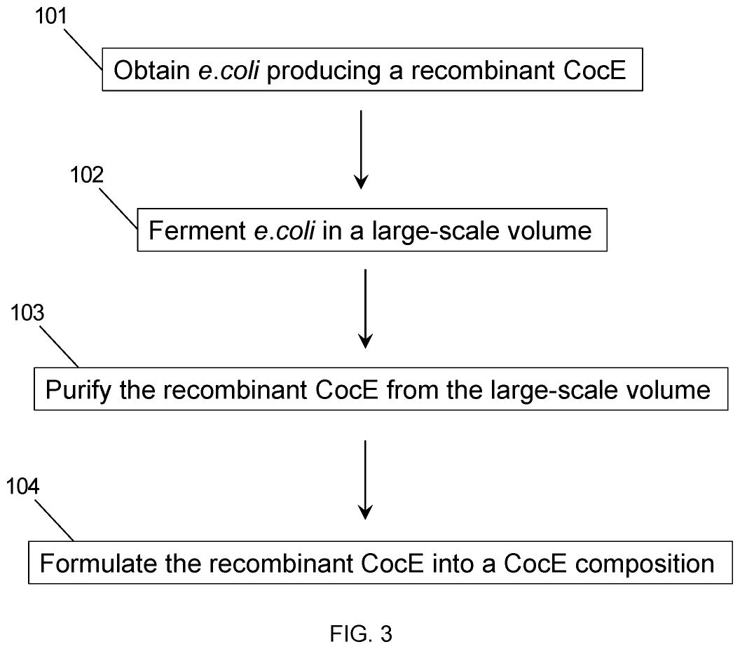

Disclosed herein are methods for manufacturing CocE compositions (FIG. 3), wherein the method comprises: a) obtaining a strain of bacteria producing a recombinant CocE 101; b) fermenting the bacteria in a large-scale volume 102; c) purifying the recombinant CocE from the large-scale volume 103; and d) formulating the recombinant CocE into a CocE composition 104.

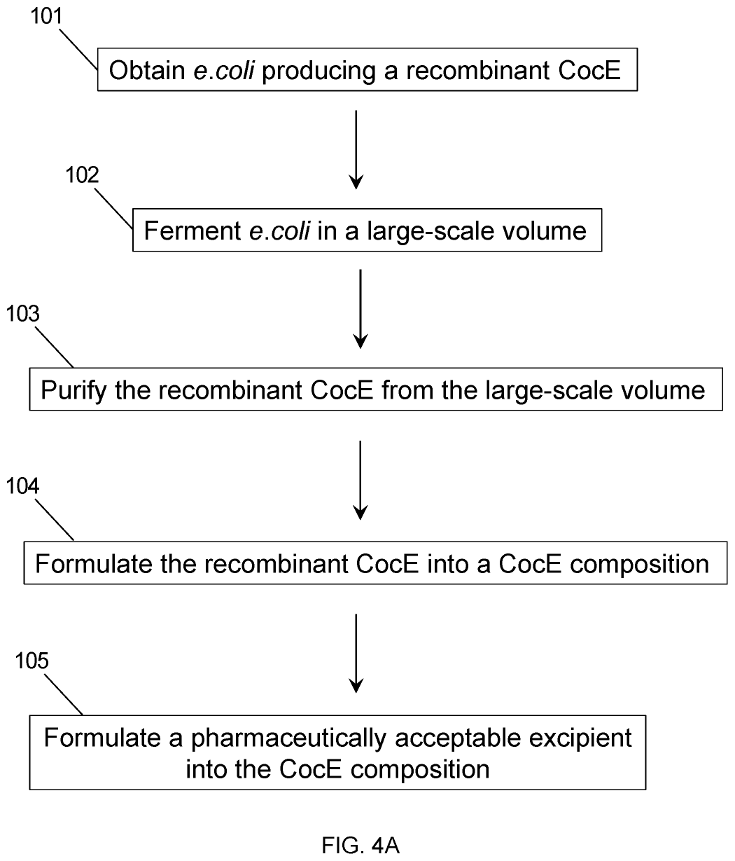

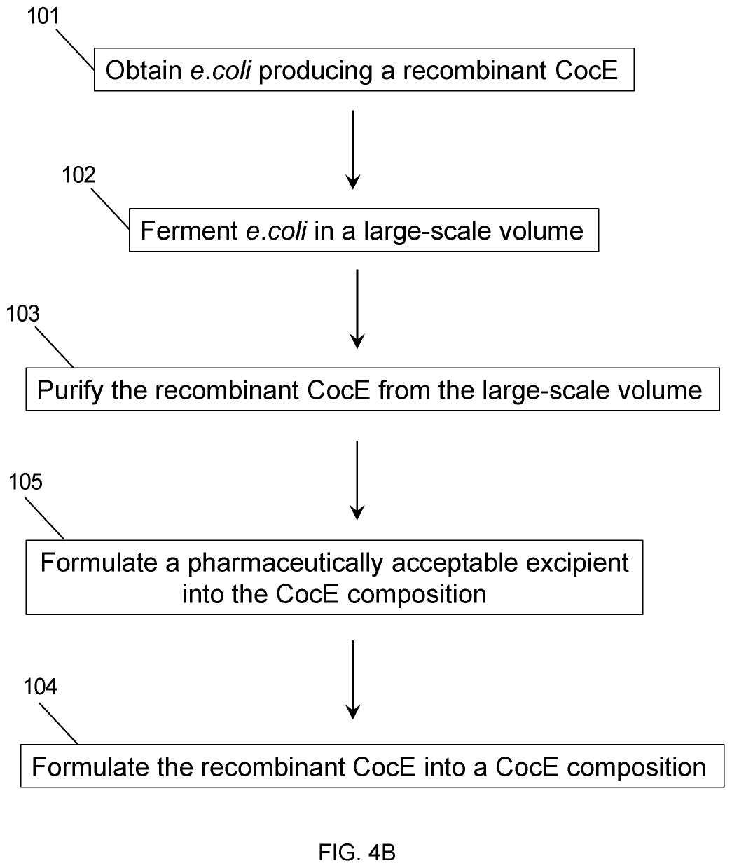

In some embodiments, disclosed herein is a method for manufacturing a CocE composition (FIGS. 4A and 4B), wherein the method comprises: a) obtaining a strain of bacteria producing a recombinant CocE 101; b) fermenting the bacteria in a large-scale volume 102; c) purifying the recombinant CocE from the large-scale volume 103; d) formulating the recombinant CocE into a CocE composition 104; and e) after step c), formulating a pharmaceutically acceptable excipient into the CocE composition 105.

In some embodiments, the strain of bacteria producing a recombinant CocE comprises E. coli, endotoxin-free E. coli, FreColi (RCT Technologies, Tucson, AZ), or a combination thereof.

### Fermentation

A fermentation of a protein or enzyme, including CocE, is influenced by a number of factors, including, temperature, pH, types of medium, dissolved O2, dissolved CO2, operational system (e.g., batch, fed-batch, continuous), feeding with precursors, mixing methods, and shear rates in the fermenter. The rate of fermentation, product yield, organoleptic properties of the product, e.g., appearance, taste, smell, texture, etc.), purity, and other physical and chemical properties vary depending on such factors.

The production of proteins or enzymes, including CocE, may be conducted in either submerged fermentation mode or solid-state mode. Submerged fermentation is the cultivation of microorganisms in liquid nutrient broth, by which industrial enzymes are commonly produced. This fermentation mode allows for microorganisms, such as, bacteria and fungi, to grow in closed vessels containing a fermentation medium and a high concentration of oxygen. The microorganisms release the desired proteins or enzymes into solution as they break down the nutrients. In solid-state fermentation mode, microorganisms cultivate on a solid substrate, such as, grains, rice, and wheat bran. This fermentation method provides advantages, including high volumetric productivity, relatively high concentration of product, less effluent generated and simple fermentation equipment.

### Fermentation Volume

In some embodiments, the specific large scale volume ranges from about 1 L to about 1,000,000 L. In some embodiments, the specific large scale volume ranges from about 1 L to about 5 L, about 1 L to about 10 L, about 1 L to about 100 L, about 1 L to about 1,000 L, about 1 L to about 10,000 L, about 1 L to about 50,000 L, about 1 L to about 70,000 L, about 1 L to about 90,000 L, about 1 L to about 100,000 L, about 1 L to about 1,000,000 L, about 5 L to about 10 L, about 5 L to about 100 L, about 5 L to about 1,000 L, about 5 L to about 10,000 L, about 5 L to about 50,000 L, about 5 L to about 70,000 L, about 5 L to about 90,000 L, about 5 L to about 100,000 L, about 5 L to about 1,000,000 L, about 10 L to about 100 L, about 10 L to about 1,000 L, about 10 L to about 10,000 L, about 10 L to about 50,000 L, about 10 L to about 70,000 L, about 10 L to about 90,000 L, about 10 L to about 100,000 L, about 10 L to about 1,000,000 L, about 100 L to about 1,000 L, about 100 L to about 10,000 L, about 100 L to about 50,000 L, about 100 L to about 70,000 L, about 100 L to about 90,000 L, about 100 L to about 100,000 L, about 100 L to about 1,000,000 L, about 1,000 L to about 10,000 L, about 1,000 L to about 50,000 L, about 1,000 L to about 70,000 L, about 1,000 L to about 90,000 L, about 1,000 L to about 100,000 L, about 1,000 L to about 1,000,000 L, about 10,000 L to about 50,000 L, about 10,000 L to about 70,000 L, about 10,000 L to about 90,000 L, about 10,000 L to about 100,000 L, about 10,000 L to about 1,000,000 L, about 50,000 L to about 70,000 L, about 50,000 L to about 90,000 L, about 50,000 L to about 100,000 L, about 50,000 L to about 1,000,000 L, about 70,000 L to about 90,000 L, about 70,000 L to about 100,000 L, about 70,000 L to about 1,000,000 L, about 90,000 L to about 100,000 L, about 90,000 L to about 1,000,000 L, or about 100,000 L to about 1,000,000 L. In some embodiments, the specific large scale volume ranges from about 1 L, about 5 L, about 10 L, about 100 L, about 1,000 L, about 10,000 L, about 50,000 L, about 70,000 L, about 90,000 L, about 100,000 L, or about 1,000,000 L. In some embodiments, the specific large scale volume ranges from at least about 1 L, about 5 L, about 10 L, about 100 L, about 1,000 L, about 10,000 L, about 50,000 L, about 70,000 L, about 90,000 L, or about 100,000 L. In some embodiments, the specific large scale volume ranges from at most about 5 L, about 10 L, about 100 L, about 1,000 L, about 10,000 L, about 50,000 L, about 70,000 L, about 90,000 L, about 100,000 L, or about 1,000,000 L. In some embodiments, the desired large scale volume ranges from 10 to 90,000 L

### Fermentation Time

In some embodiments, the fermentation period for recombinant CocE production ranges from 4 hours to a few days. In some embodiments, the fermentation period for recombinant CocE production ranges from 4 hours to 5 days. In some embodiments, the fermentation period for recombinant CocE production ranges from 4 hours to 4 days. In some embodiments, the fermentation period for recombinant CocE production ranges from 4 hours to 3 days. In some embodiments, the fermentation period for recombinant CocE production ranges from 4 hours to 2 days. In some embodiments, the fermentation period for recombinant CocE production ranges from 4 hours to 1 day. In some embodiments, for instance, the fermentation period for recombinant CocE production is 5 days, during which 1 day on a plate, 0.5 day in a 50 mL culture, 0.25 day in a 1 L culture, 0.25 day in a 30 L culture, 0.5 day in a 500 L culture, and 1 day in a 90,000 L culture, in a consecutive order.

### Fermentation Temperature

In some embodiments, the fermentation temperature for recombinant CocE production ranges from 6 degrees Celsius to 37 degrees Celsius.

### Purification Methods

Various purification methods for proteins or enzymes, including recombinant CocE, may be applied in sequence to attain high purity levels. When the purification volume is large, it is recommended to use less expensive and simple methods at early stages followed by more expensive and complex methods when the volume remaining is small. It is ideal for the purification to result in high final degree of purity, high overall recovery of protein or enzyme activity, and reproducibility. Examples of existing purification methods include, but not limited to, salting out, extraction, precipitation, dialysis, filtration, chromatography, centrifugation, or a combination thereof.

In some embodiments, purifying the recombinant cocaine esterase from the large-scale volume comprises extraction, centrifugation, immobilized metal chromatography, ion exchange chromatography, size exclusion chromatography, or a combination thereof.

### Purification Volume

In some embodiments, a yield of purified recombinant CocE after purifying the recombinant cocaine esterase from the large-scale volume ranges from about 50% to about 99.99%. In some embodiments, a yield of purified recombinant CocE after purifying the recombinant cocaine esterase from the large scale volume ranges from about 50% to about 60%, about 50% to about 70%, about 50% to about 80%, about 50% to about 90%, about 50% to about 91%, about 50% to about 93%, about 50% to about 95%, about 50% to about 97%, about 50% to about 99%, about 50% to about 99.9%, about 50% to about 99.99%, about 60% to about 70%, about 60% to about 80%, about 60% to about 90%, about 60% to about 91%, about 60% to about 93%, about 60% to about 95%, about 60% to about 97%, about 60% to about 99%, about 60% to about 99.9%, about 60% to about 99.99%, about 70% to about 80%, about 70% to about 90%, about 70% to about 91%, about 70% to about 93%, about 70% to about 95%, about 70% to about 97%, about 70% to about 99%, about 70% to about 99.9%, about 70% to about 99.99%, about 80% to about 90%, about 80% to about 91%, about 80% to about 93%, about 80% to about 95%, about 80% to about 97%, about 80% to about 99%, about 80% to about 99.9%, about 80% to about 99.99%, about 90% to about 91%, about 90% to about 93%, about 90% to about 95%, about 90% to about 97%, about 90% to about 99%, about 90% to about 99.9%, about 90% to about 99.99%, about 91% to about 93%, about 91% to about 95%, about 91% to about 97%, about 91% to about 99%, about 91% to about 99.9%, about 91% to about 99.99%, about 93% to about 95%, about 93% to about 97%, about 93% to about 99%, about 93% to about 99.9%, about 93% to about 99.99%, about 95% to about 97%, about 95% to about 99%, about 95% to about 99.9%, about 95% to about 99.99%, about 97% to about 99%, about 97% to about 99.9%, about 97% to about 99.99%, about 99% to about 99.9%, about 99% to about 99.99%, or about 99.9% to about 99.99%. In some embodiments, a yield of purified recombinant CocE after purifying the recombinant cocaine esterase from the large-scale volume ranges from about 50%, about 60%, about 70%, about 80%, about 90%, about 91%, about 93%, about 95%, about 97%, about 99%, about 99.9%, or about 99.99%. In some embodiments, a yield of purified recombinant CocE after purifying the recombinant cocaine esterase from the large-scale volume ranges from at least about 50%, about 60%, about 70%, about 80%, about 90%, about 91%, about 93%, about 95%, about 97%, about 99%, or about 99.9%. In some embodiments, a yield of purified recombinant CocE after purifying the recombinant cocaine esterase from the large-scale volume ranges from at most about 60%, about 70%, about 80%, about 90%, about 91%, about 93%, about 95%, about 97%, about 99%, about 99.9%, or about 99.99%. In some embodiments, the purity of the compositions disclosed herein ranges from 70% to 99%. In some embodiments, the purity of the compositions disclosed herein ranges from about 50% to about 60%, about 50% to about 70%, about 50% to about 80%, about 50% to about 90%, about 50% to about 91%, about 50% to about 93%, about 50% to about 95%, about 50% to about 97%, about 50% to about 99%, about 50% to about 99.9%, about 50% to about 99.99%, about 60% to about 70%, about 60% to about 80%, about 60% to about 90%, about 60% to about 91%, about 60% to about 93%, about 60% to about 95%, about 60% to about 97%, about 60% to about 99%, about 60% to about 99.9%, about 60% to about 99.99%, about 70% to about 80%, about 70% to about 90%, about 70% to about 91%, about 70% to about 93%, about 70% to about 95%, about 70% to about 97%, about 70% to about 99%, about 70% to about 99.9%, about 70% to about 99.99%, about 80% to about 90%, about 80% to about 91%, about 80% to about 93%, about 80% to about 95%, about 80% to about 97%, about 80% to about 99%, about 80% to about 99.9%, about 80% to about 99.99%, about 90% to about 91%, about 90% to about 93%, about 90% to about 95%, about 90% to about 97%, about 90% to about 99%, about 90% to about 99.9%, about 90% to about 99.99%, about 91% to about 93%, about 91% to about 95%, about 91% to about 97%, about 91% to about 99%, about 91% to about 99.9%, about 91% to about 99.99%, about 93% to about 95%, about 93% to about 97%, about 93% to about 99%, about 93% to about 99.9%, about 93% to about 99.99%, about 95% to about 97%, about 95% to about 99%, about 95% to about 99.9%, about 95% to about 99.99%, about 97% to about 99%, about 97% to about 99.9%, about 97% to about 99.99%, about 99% to about 99.9%, about 99% to about 99.99%, or about 99.9% to about 99.99%.

### Formulations

Protein or enzyme formulation is an important process step following production and purification. Disclosure herein relates to a CocE composition comprising a recombinant CocE. In some embodiments, the compositions, methods, and articles disclosed herein comprise a CocE composition comprising a recombinant CocE and oxime compounds. In some embodiments, the compositions, methods, and articles disclosed herein comprise a recombinant CocE, an oxime compound, and a pharmaceutically acceptable excipient.

In some embodiments, the present disclosure comprises a composition having, by weight, 0.1-20% of a CocE and 0.01-2% of an oxime compound. In some embodiments, the present disclosure comprises a composition having, by weight, 0.2-18% of a CocE and 0.02-1.8% of an oxime compound. In some embodiments, the present disclosure comprises a composition having, by weight, 0.3-16% of a CocE and 0.03-1.6% of an oxime compound. In some embodiments, the present disclosure comprises a composition having, by weight, 0.4-14% of a CocE and 0.04-1.4% of an oxime compound. In some embodiments, the present disclosure comprises a composition having, by weight, 0.4-12% of a CocE and 0.04-1.2% of an oxime compound. In some embodiments, the present disclosure comprises a composition having, by weight, 0.5-10% of a CocE and 0.05-1% of an oxime compound.

In some embodiments, the present disclosure comprises a composition having, by weight, 1% of Tris-HCl and up to 5% of NaCl, wherein Tris-HCl and NaCl are pharmaceutically acceptable excipients. In some embodiments, the present disclosure comprises a composition having, by weight, 0.9% of Tris-HCl and up to 4% of NaCl, wherein Tris-HCl and NaCl are pharmaceutically acceptable excipients. In some embodiments, the present disclosure comprises a composition having, by weight, 0.8% of Tris-HCl and up to 3.5% of NaCl, wherein Tris-HCl and NaCl are pharmaceutically acceptable excipients. In some embodiments, the present disclosure comprises a composition having, by weight, 0.7% of Tris-HCl and up to 3% of NaCl, wherein Tris-HCl and NaCl are pharmaceutically acceptable excipients. In some embodiments, the present disclosure comprises a composition having, by weight, 0.6% of Tris-HCl and up to 2.6% of NaCl, wherein Tris-HCl and NaCl are pharmaceutically acceptable excipients.

In some embodiments, the specific amount of the CocE in the pharmaceutically acceptable excipient ranges, by weight, from about 0.1% to about 20%. In some embodiments, the specific amount of the CocE in the pharmaceutically acceptable excipient ranges from about 0.1% to about 0.2%, about 0.1% to about 0.3%, about 0.1% to about 0.4%, about 0.1% to about 0.5%, about 0.1% to about 1%, about 0.1% to about 3%, about 0.1% to about 5%, about 0.1% to about 7%, about 0.1% to about 10%, about 0.1% to about 15%, about 0.1% to about 20%, about 0.2% to about 0.3%, about 0.2% to about 0.4%, about 0.2% to about 0.5%, about 0.2% to about 1%, about 0.2% to about 3%, about 0.2% to about 5%, about 0.2% to about 7%, about 0.2% to about 10%, about 0.2% to about 15%, about 0.2% to about 20%, about 0.3% to about 0.4%, about 0.3% to about 0.5%, about 0.3% to about 1%, about 0.3% to about 3%, about 0.3% to about 5%, about 0.3% to about 7%, about 0.3% to about 10%, about 0.3% to about 15%, about 0.3% to about 20%, about 0.4% to about 0.5%, about 0.4% to about 1%, about 0.4% to about 3%, about 0.4% to about 5%, about 0.4% to about 7%, about 0.4% to about 10%, about 0.4% to about 15%, about 0.4% to about 20%, about 0.5% to about 1%, about 0.5% to about 3%, about 0.5% to about 5%, about 0.5% to about 7%, about 0.5% to about 10%, about 0.5% to about 15%, about 0.5% to about 20%, about 1% to about 3%, about 1% to about 5%, about 1% to about 7%, about 1% to about 10%, about 1% to about 15%, about 1% to about 20%, about 3% to about 5%, about 3% to about 7%, about 3% to about 10%, about 3% to about 15%, about 3% to about 20%, about 5% to about 7%, about 5% to about 10%, about 5% to about 15%, about 5% to about 20%, about 7% to about 10%, about 7% to about 15%, about 7% to about 20%, about 10% to about 15%, about 10% to about 20%, or about 15% to about 20%. In some embodiments, the specific amount of the CocE in the pharmaceutically acceptable excipient ranges from about 0.1%, about 0.2%, about 0.3%, about 0.4%, about 0.5%, about 1%, about 3%, about 5%, about 7%, about 10%, about 15%, or about 20%. In some embodiments, the specific amount of the CocE in the pharmaceutically acceptable excipient ranges from at least about 0.1%, about 0.2%, about 0.3%, about 0.4%, about 0.5%, about 1%, about 3%, about 5%, about 7%, about 10%, or about 15%. In some embodiments, the specific amount of the CocE in the pharmaceutically acceptable excipient ranges from at most about 0.2%, about 0.3%, about 0.4%, about 0.5%, about 1%, about 3%, about 5%, about 7%, about 10%, about 15%, or about 20%. In some embodiments, the desired amount of the CocE in the pharmaceutically acceptable excipient ranges from 0.5 to 10%.

In some embodiments, the specific amount of the oxime compounds in the pharmaceutically acceptable excipient ranges, by weight, from about 0.001% to about 5%. In some embodiments, the specific amount of the oxime compounds in the pharmaceutically acceptable excipient ranges, by weight, from about 0.001% to about 0.01%, about 0.001% to about 0.02%, about 0.001% to about 0.03%, about 0.001% to about 0.04%, about 0.001% to about 0.05%, about 0.001% to about 1%, about 0.001% to about 2%, about 0.001% to about 3%, about 0.001% to about 4%, about 0.001% to about 5%, about 0.01% to about 0.02%, about 0.01% to about 0.03%, about 0.01% to about 0.04%, about 0.01% to about 0.05%, about 0.01% to about 1%, about 0.01% to about 2%, about 0.01% to about 3%, about 0.01% to about 4%, about 0.01% to about 5%, about 0.02% to about 0.03%, about 0.02% to about 0.04%, about 0.02% to about 0.05%, about 0.02% to about 1%, about 0.02% to about 2%, about 0.02% to about 3%, about 0.02% to about 4%, about 0.02% to about 5%, about 0.03% to about 0.04%, about 0.03% to about 0.05%, about 0.03% to about 1%, about 0.03% to about 2%, about 0.03% to about 3%, about 0.03% to about 4%, about 0.03% to about 5%, about 0.04% to about 0.05%, about 0.04% to about 1%, about 0.04% to about 2%, about 0.04% to about 3%, about 0.04% to about 4%, about 0.04% to about 5%, about 0.05% to about 1%, about 0.05% to about 2%, about 0.05% to about 3%, about 0.05% to about 4%, about 0.05% to about 5%, about 1% to about 2%, about 1% to about 3%, about 1% to about 4%, about 1% to about 5%, about 2% to about 3%, about 2% to about 4%, about 2% to about 5%, about 3% to about 4%, about 3% to about 5%, or about 4% to about 5%. In some embodiments, the specific amount of the oxime compounds in the pharmaceutically acceptable excipient ranges, by weight, from about 0.001%, about 0.01%, about 0.02%, about 0.03%, about 0.04%, about 0.05%, about 1%, about 2%, about 3%, about 4%, or about 5%. In some embodiments, the specific amount of the oxime compounds in the pharmaceutically acceptable excipient ranges, by weight, from at least about 0.001%, about 0.01%, about 0.02%, about 0.03%, about 0.04%, about 0.05%, about 1%, about 2%, about 3%, or about 4%. In some embodiments, amount concentration of the oxime compounds in the pharmaceutically acceptable excipient ranges, by weight, from at most about 0.01%, about 0.02%, about 0.03%, about 0.04%, about 0.05%, about 1%, about 2%, about 3%, about 4%, or about 5%. In some embodiments, desired amount of the oxime compounds in the pharmaceutically acceptable excipient ranges, by weight, from 0.05 to 1%.

In some embodiments, after fermentation or purification process, the cocaine esterase is further PEGylated. Particularly, covalent and non-covalent attachment of a PEG molecule to biological molecules, such as proteins and enzymes, such as CocE, called PEGylation, can improve pharmacokinetics of such biological molecules by increasing the molecular mass of proteins and peptides and shielding them from proteases. Each PEG segment can combine with two or three molecules, making the overall compound larger and more hydrophilic. The PEG structure may be either linear or branched, and the branched PEG tends to increase in vivo half-life by increasing “stealth” properties of a conjugated biological molecules. Also, PEGylation allows to modify physiological properties and prolong the retention of the therapeutic agents in the body.

The introduction of different functional groups to the end of a PEG molecule allows for more site-specific reactions. For instance, various amino acid residue in proteins may get involved in chemical reactions with PEG having amine, sulfhydryl, carboxyl, and carbonyl groups. By altering the chain-end functional group of the PEG molecules, it may get easier to target these amino acid residues or specific functional groups. Examples of chain-end functional groups include, but not limited to, carboxyl, N-hydroxysuccinimide (NHS), anhydride, ester, aminooxy, amino, alkyne, azide, bicyclo[6.1.0] nonyne (BCN), dibenzocyclooctyne (DBCO), trans-cyclooctene (TCO), tetrazine, bromo, or a combination thereof.

In some embodiments, molecular weight of linear PEG ranges from about 100 to about 1,000,000 g/mol. In some embodiments, molecular weight of linear PEG ranges from about 100 to about 800,000 g/mol. In some embodiments, molecular weight of linear PEG ranges from about 100 to about 500,000 g/mol. In some embodiments, molecular weight of linear PEG ranges from about 100 to about 100,000 g/mol. In some embodiments, molecular weight of linear PEG ranges from about 100 to about 50,000 g/mol. In some embodiments, molecular weight of linear PEG ranges from about 100 to about 40,000 g/mol. In some embodiments, molecular weight of linear PEG ranges from about 100 to about 30,000 g/mol. In some embodiments, molecular weight of linear PEG ranges from about 500 to about 30,000 g/mol. In some embodiments, molecular weight of linear PEG ranges from about 1,000 to about 30,000 g/mol. In some embodiments, molecular weight of linear PEG ranges from about 2,000 to about 30,000 g/mol. In some embodiments, molecular weight of linear PEG ranges from about 3,000 to about 30,000 g/mol. In some embodiments, molecular weight of linear PEG ranges from about 4,000 to about 30,000 g/mol. In some embodiments, molecular weight of linear PEG ranges from about 4,000 to about 25,000 g/mol. In some embodiments, molecular weight of linear PEG ranges from about 5,000 to about 25,000 g/mol. In some embodiments, molecular weight of linear PEG ranges from about 5,000 to about 20,000 g/mol. In some embodiments, molecular weight of linear PEG ranges from about 100 to about 500 g/mol. In some embodiments, the desired molecular weight of linear PEG ranges from about 5,000 to about 20,000 g/mol.

In some embodiments, molecular weight of branched PEG ranges from about 100 to about 1,000,000 g/mol. In some embodiments, molecular weight of branched PEG ranges from about 1,000 to about 500,000 g/mol. In some embodiments, molecular weight of branched PEG ranges from about 5,000 to about 100,000 g/mol. In some embodiments, molecular weight of branched PEG ranges from about 10,000 to about 80,000 g/mol. In some embodiments, molecular weight of branched PEG ranges from about 12,000 to about 70,000 g/mol. In some embodiments, molecular weight of branched PEG ranges from about 13,000 to about 60,000 g/mol. In some embodiments, molecular weight of branched PEG ranges from about 14,000 to about 55,000 g/mol. In some embodiments, molecular weight of branched PEG ranges from about 15,000 to about 50,000 g/mol. In some embodiments, molecular weight of branched PEG ranges from about 16,000 to about 48,000 g/mol. In some embodiments, molecular weight of branched PEG ranges from about 17,000 to about 46,000 g/mol. In some embodiments, molecular weight of branched PEG ranges from about 18,000 to about 44,000 g/mol. In some embodiments, molecular weight of branched PEG ranges from about 19,000 to about 42,000 g/mol. In some embodiments, molecular weight of branched PEG ranges from about 20,000 to about 40,000 g/mol. In some embodiments, the desired molecular weight of branched PEG ranges from about 20,000 to about 40,000 g/mol.

In some embodiments, the recombinant CocE is active for greater than or equal to 1 h at 37° C. In some embodiments, the recombinant CocE is active for greater than or equal to 2 h at 37° C. In some embodiments, the recombinant CocE is active for greater than or equal to 3 h at 37° C. In some embodiments, the recombinant CocE is active for greater than or equal to 4 h at 37° C. In some embodiments, the recombinant CocE is active for greater than or equal to 5 h at 37° C. In some embodiments, the recombinant CocE is active for greater than or equal to 6 h at 37° C.

In some embodiments, maximum initial velocity of a reaction (Vₘₐₓ) of the recombinant CocE in the composition ranges, for organophosphate-based agents, from about 10 μmol/min to about 50,000 μmol/min. In some embodiments, Vₘₐₓ of the recombinant CocE in the composition ranges, for organophosphate-based agents, from about 10 μmol/min to about 100 umol/min, about 10 μmol/min to about 1,000 μmol/min, about 10 μmol/min to about 2,000 μmol/min, about 10 μmol/min to about 4,000 μmol/min, about 10 μmol/min to about 6,000 μmol/min, about 10 μmol/min to about 8,000 μmol/min, about 10 μmol/min to about 10,000 μmol/min, about 10 μmol/min to about 12,000 μmol/min, about 10 μmol/min to about 15,000 μmol/min, about 10 μmol/min to about 20,000 μmol/min, about 10 μmol/min to about 50,000 μmol/min, about 100 μmol/min to about 1,000 μmol/min, about 100 μmol/min to about 2,000 μmol/min, about 100 μmol/min to about 4,000 μmol/min, about 100 μmol/min to about 6,000 μmol/min, about 100 μmol/min to about 8,000 μmol/min, about 100 μmol/min to about 10,000 μmol/min, about 100 μmol/min to about 12,000 μmol/min, about 100 μmol/min to about 15,000 μmol/min, about 100 μmol/min to about 20,000 μmol/min, about 100 μmol/min to about 50,000 μmol/min, about 1,000 μmol/min to about 2,000 μmol/min, about 1,000 μmol/min to about 4,000 μmol/min, about 1,000 μmol/min to about 6,000 μmol/min, about 1,000 μmol/min to about 8,000 μmol/min, about 1,000 μmol/min to about 10,000 μmol/min, about 1,000 μmol/min to about 12,000 μmol/min, about 1,000 μmol/min to about 15,000 μmol/min, about 1,000 μmol/min to about 20,000 μmol/min, about 1,000 μmol/min to about 50,000 μmol/min, about 2,000 μmol/min to about 4,000 μmol/min, about 2,000 μmol/min to about 6,000 μmol/min, about 2,000 μmol/min to about 8,000 μmol/min, about 2,000 μmol/min to about 10,000 μmol/min, about 2,000 μmol/min to about 12,000 μmol/min, about 2,000 μmol/min to about 15,000 μmol/min, about 2,000 μmol/min to about 20,000 μmol/min, about 2,000 μmol/min to about 50,000 μmol/min, about 4,000 μmol/min to about 6,000 μmol/min, about 4,000 μmol/min to about 8,000 μmol/min, about 4,000 μmol/min to about 10,000 μmol/min, about 4,000 μmol/min to about 12,000 μmol/min, about 4,000 μmol/min to about 15,000 μmol/min, about 4,000 μmol/min to about 20,000 μmol/min, about 4,000 μmol/min to about 50,000 μmol/min, about 6,000 μmol/min to about 8,000 μmol/min, about 6,000 μmol/min to about 10,000 μmol/min, about 6,000 μmol/min to about 12,000 μmol/min, about 6,000 μmol/min to about 15,000 μmol/min, about 6,000 μmol/min to about 20,000 μmol/min, about 6,000 μmol/min to about 50,000 μmol/min, about 8,000 μmol/min to about 10,000 μmol/min, about 8,000 μmol/min to about 12,000 μmol/min, about 8,000 μmol/min to about 15,000 μmol/min, about 8,000 μmol/min to about 20,000 μmol/min, about 8,000 μmol/min to about 50,000 μmol/min, about 10,000 μmol/min to about 12,000 μmol/min, about 10,000 μmol/min to about 15,000 μmol/min, about 10,000 μmol/min to about 20,000 μmol/min, about 10,000 μmol/min to about 50,000 μmol/min, about 12,000 μmol/min to about 15,000 μmol/min, about 12,000 μmol/min to about 20,000 μmol/min, about 12,000 μmol/min to about 50,000 μmol/min, about 15,000 μmol/min to about 20,000 μmol/min, about 15,000 μmol/min to about 50,000 μmol/min, or about 20,000 μmol/min to about 50,000 μmol/min. In some embodiments, Vₘₐₓ of the recombinant CocE in the composition ranges, for organophosphate-based agents, from about 10 μmol/min, about 100 μmol/min, about 1,000 μmol/min, about 2,000 μmol/min, about 4,000 μmol/min, about 6,000 μmol/min, about 8,000 μmol/min, about 10,000 μmol/min, about 12,000 μmol/min, about 15,000 μmol/min, about 20,000 μmol/min, or about 50,000 μmol/min. In some embodiments, Vₘₐₓ of the recombinant CocE in the composition ranges, for organophosphate-based agents, from at least about 10 μmol/min, about 100 μmol/min, about 1,000 μmol/min, about 2,000 μmol/min, about 4,000 μmol/min, about 6,000 μmol/min, about 8,000 μmol/min, about 10,000 μmol/min, about 12,000 μmol/min, about 15,000 μmol/min, or about 20,000 μmol/min. In some embodiments, Vₘₐₓ of the recombinant CocE in the composition ranges, for organophosphate-based agents, from at most about 100 μmol/min, about 1,000 μmol/min, about 2,000 μmol/min, about 4,000 μmol/min, about 6,000 μmol/min, about 8,000 μmol/min, about 10,000 μmol/min, about 12,000 μmol/min, about 15,000 μmol/min, about 20,000 μmol/min, or about 50,000 μmol/min. In yet another exemplary embodiment, Vₘₐₓ of the recombinant CocE in the composition, for cocaine, resulted in a range from 1200 to 12,000 μmol/min.

### Articles of Personal Protection Equipment Comprising a CocE

According to the United States Department of Labor, personal protective equipment (PPE) is equipment worn to minimize exposure to hazards that cause serious workplace injuries and illnesses from contact with chemical, radiological, physical, electrical, mechanical, or other workplace hazards. PPE may include items, such as, gloves, safety glasses and shoes, earplugs or muffs, hard hats, respirators, coveralls, vests, full body suits, etc.

In some embodiments, the present invention relates to an article of PPE that comprises a CocE for protecting a wearer against exposure to chemical weapons comprising nerve agents or organophosphate-based nerve agents. In some embodiments, the present invention relates to an article of PPE that comprises a CocE for protecting a wearer against exposure to organophosphate pesticides. In some embodiments, the present invention relates to an article of PPE that comprises a CocE for protecting a wearer against exposure to chemical weapons, comparing nerve agents or organophosphate-based nerve agents, organophosphate pesticides, or a combination thereof.

Designing the proper PPE to minimize exposure to hazards, such as chemical weapons and organophosphate pesticides, is essential to safety depending on the hazard or workplace conditions. For the purpose of this invention, required PPE may include eye and face protection, hand protection, body protection, respiratory protection, and hearing protection. Particularly, a person may be exposed to chemical weapons and organophosphate pesticides by skin and eye contact, inhaling hazardous vapors, and swallowing.

In some embodiments, a durable footwear or a footwear cover can protect against such chemicals spilled. In some embodiments, gloves, including chemical-resistant gloves and insulated gloves, can protect a person upon exposure to such chemicals. The chemical-resistant gloves may be made from natural rubber, neoprene, nitrile, poly(vinyl chloride) (PVC), polyethylene, or other existing substances to protect against such chemicals. Aprons, coats, gowns, jackets, pants, and full body suits may be worn for body protection, and they may be made from rubber, leather, or synthetics. In some embodiments, body protection may be ensured by wearing a traditional cotton/cotton-polyester blend coat, a flame-resistant coat (e.g., Nomex or other flame-resistant cotton), or barrier suit or suit. In some embodiments, exposure to air contaminated with gases, vapors, fumes, sprays, dusts, fogs, mists, or smoke of chemicals disclosed herein may be minimized by wearing respirators. Effective respirators cover the nose and mouth, a wearer's entire face, or the entire head. In some embodiments, eye and face protection can be achieved by wearing eyewear specifically designed to reduce the risk of exposure to chemical splashes, laser radiation, or flying debris. PPEs for such eye and face protection include general safety glasses, laser safety glasses, chemical splash goggles, impact goggles, and face shields. In some embodiments, materials for PPE may be fabrics, plastics, rubbers, metals, or a combination thereof.

In some embodiments, the present invention relates to an article of PPE comprising a CocE, wherein the CocE is incorporated into the PPE during a manufacturing step. In some embodiments, the present invention relates to a respirator configured to an accessory, such as a removable container (or canister), wherein inner surface of the accessory is coated with CocE for detoxifying an organophosphate-based agent. In some embodiments, the present invention relates to a respirator configured to an accessory, such as a removable container (or canister), wherein the accessory is filled with CocE for detoxifying an organophosphate-based agent. For instance, the accessory is filled with a matrix, such as, a chromatography resin, to which CocE is coated chemically or physically. In FIG. 11, a container of CocE 504 is configured to a respirator composing a strap 501, a respirator body 502, and a filter cartridge 503.

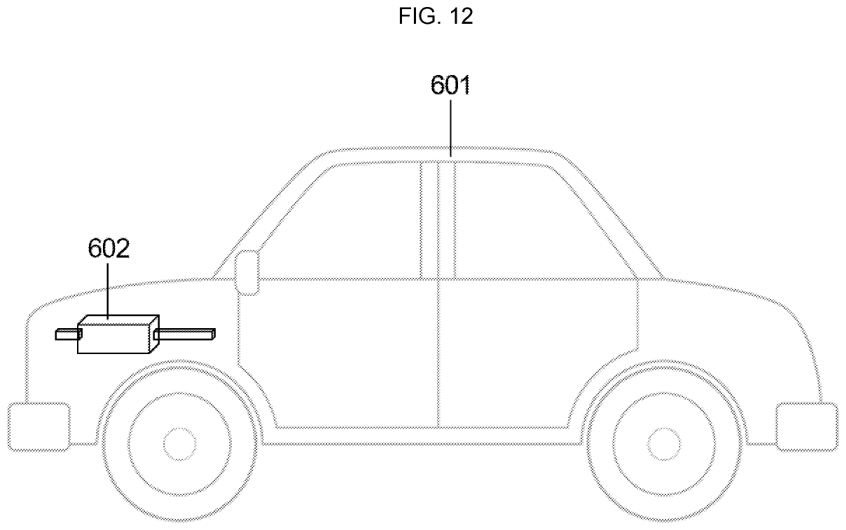

In some embodiments, the present invention relates to an air intake system 602 of a vehicle 601 that can prevent the introduction of an organophosphate-based agent into the interior of the vehicle or release CocE into the interior of the vehicle where a driver and a passenger(s) are located so that the released CocE can detoxify an organophosphate-based agent (FIG. 12). In some embodiments, surface of the air intake system is coated with CocE for detoxifying an organophosphate-based agent. In some embodiments, the air intake system is filled with CocE for detoxifying an organophosphate-based agent. For instance, the air intake system is filled with a chromatography resin to which CocE is coated chemically or physically.

### Definitions

Unless otherwise defined, all of the technical terms used herein have the same meaning as commonly understood by one of ordinary skill in the art in the field to which this disclosure belongs.

As used in this specification and the appended claims, the singular forms “a”, “an”, and “the” include plural references unless the context clearly dictates otherwise. Any reference to “or” herein is intended to encompass “and/or” unless otherwise stated.

As used herein, the terms “comprising” (and any form or variant of comprising, such as “comprise” and “comprises”), “having” (and any form or variant of having, such as “have” and “has”), “including” (and any form or variant of including, such as “includes” and “include”), or “containing” (and any form or variant of containing, such as “contains” and “contain”), are inclusive or open-ended and do not exclude additional, unrecited additives, components, integers, elements or method steps.

As used herein, the term “about” a number refers to that number plus or minus 10% of that number. The term ‘about’ when used in the context of a range refers to that range minus 10% of its lowest value and plus 10% of its greatest value.

Whenever the term “at least”, “greater than”, or “greater than or equal to” precedes the first numerical value in a series of two or more numerical values, the term “at least”, “greater than,” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than”, “less than”, or “less than or equal to” precedes the first numerical value in a series of two or more numerical values, the term “no more than”, “less than”, or “less than or equal to” applies to each of the numerical values in that series of numerical values. For example, less than or equal to 3, 2, or 1 is equivalent to less than or equal to 3, less than or equal to 2, or less than or equal to 1.

The phrase “one or more pharmaceutically acceptable excipients” is used herein to refer that one pharmaceutically acceptable excipient or more than one pharmaceutically acceptable excipient may be used in any combination. The number of pharmaceutically acceptable excipients to be used may be at the discretion of a person skilled in the art, and they may be of different types.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more.

The term “specific pH” herein refers to a desired pH value of a solvent or a solution comprising CocE obtained by adding a pharmaceutically acceptable excipient.

The term “% wt” is used to describe the weight percentage of one component in a mixture of components.

The term “a trace” herein refers more than, but close to about 0%.

The term “about” herein refers to +10%, +20%, +30%, +40%, or +50%, or to the nearest significant figure.

The term “specific ratio” herein refers to a weight ratio between a CocE and an oxime compound present in a recombinant CocE composition. The ratio may be altered by a person of skill in the art according to preference.

The term “desired amount” herein refers to an amount of a CocE or an oxime compound in a recombinant CocE composition. The amount of each of these components is controlled by the process for formulating the recombinant CocE composition. The amount may be altered by a person of skill in the art according to preference. The amount is a percentage ratio by weight that may be accurate up to two significant figures.

The term “dose” herein refers to a quantity of a medicine or drug, including CocE and CocE composition disclosed herein, taken or recommended to be taken at a particular time.

The term “purification” may be used herein to refer to a process for obtaining a purified CocE. The purification process is a separate process from the formulation process.

The term “formulation” as used herein refers to a process of obtaining a CocE composition that has a defined percentage content of CocE.

As used herein, the expression “formulating” the recombinant CocE into a CocE composition refers to adding a recombinant CocE to a CocE composition to obtain a mixture with a specific, total concentration of CocE, by weight. The mixture may then be further formulated with a pharmaceutically acceptable excipient, e.g., solvent, to form a solution composition with a pre-calculated percentage concentration by weight of CocE. The total amount of CocE content may be specified to an accuracy of up to three significant figures.

The term “composition”, “CocE composition”, “recombinant CocE composition”, or “formulated recombinant CocE composition” is used herein to describe a composition, including a CocE or a purified CocE, which has been standardized by the addition of oxime compounds or pharmaceutically acceptable excipients according to a presently described process. The standardized recombinant CocE composition includes the CocE in a specific amount.

As used herein, the term “specific amount” when referring to a CocE content means a desired percentage, accurate to one or two decimal places or one or two significant figures, of the CocE content in a CocE composition. The specific amount is defined as a percentage by weight and can be selected by a person of skill in the art according to preference.

The term “excipient” means any component added to a CocE to make a composition. An excipient is inert in relation to the CocE, in that it essentially does not act in the same way as the CocE. An excipient may be completely inert, or it may have some other property that protects the integrity of the active ingredient or assists its uptake into the human body. There are multiple types of excipient, each having a different purpose, and a given excipient may fulfill more than one purpose. Examples of types of excipient include solvents, flowability agents, flavorants, colorants, palatants, antioxidants, bioavailability-increasing agents, viscosity modifying agents, tonicity agents, drug carriers, sustained-release agents, comfort-enhancing agents, emulsifiers, solubilizing aids, lubricants, binding agents and stabilizing agents. Specific excipients include solvents, comprising water, organic solvent, or a combination thereof.

The term “purified water” includes deionized water, distilled water, reverse osmosis water, or otherwise purified water which is substantially without free ions.

The term “therapeutic effects” is intended to qualify the amount of CocE required in the treatment of a disease or disorder or on the effecting of a clinical endpoint. Reference to “treatment” of a patient is intended to include detoxifying an organophosphate-based agent. Treatment may also be preemptive in nature, i.e., it may include prevention of disease. Prevention of a disease may involve complete protection from disease, for example as in the case of prevention of intoxication with an organophosphate-based agent or may involve prevention of intoxication progression. For example, prevention of an intoxication may not mean complete foreclosure of any effect related to the intoxication at any level, but instead may mean prevention of the symptoms of an intoxication to a clinically significant or detectable level. Prevention of intoxication may also mean prevention of progression of an intoxication to a later stage of the disease.

In some embodiments, as the ranges become narrower and more central compared to the greatest range, the properties of the embodiments generally become more balanced, such properties being solubility, viscosity, flowability, stability, taste, potency, immediate potency, delayed potency, cost of production, efficiency of purification, efficiency of formulation, efficiency of production, purification time, formulation time, production time, compatibility of the recombinant CocE composition, therapeutic efficacy of the CocE, psychoactive efficacy of the recombinant CocE composition, and so on. As the ranges become narrower towards one extreme or other of the widest range, a particular property of the composition or process becomes more pronounced relative to the other properties. The specific range is to be chosen depending on how the properties are to be traded-off against each other.

### Examples

These examples are provided for illustrative purposes only and not to limit the scope of the claims provided herein.

### Test System

The experiments were conducted using the candidate MCM, human AChE, and dilute sarin. The AChE source was erythrocyte membrane preparations derived from human whole blood. Sarin concentrations in solution were evaluated by liquid chromatography with tandem mass spectrometry (LC-MS/MS), and AChE activity was evaluated using a spectrophotometric assay.

### Production of Thermostabilized Mutant of CocE

A highly active, thermostabilized mutant of CocE was produced by Genscript.

Methods for expression and purification of CocE was well established in the literature (N. Aurbek, H. Thiermann, F. Eyer, P. Eyer, F. Worek, Suitability of human butyrylcholinesterase as therapeutic marker and pseudo catalytic scavenger in organophosphate poisoning: A kinetic analysis. Toxicology 259, 133-139 (2009); T.-M. Shih, J. A. Guarisco, T. M. Myers, R. K. Kan, J. H. McDonough. The oxime pro-2-PAM provides minimal protection against the CNS effects of the nerve agents sarin, cyclosarin, and VX in guinea pigs. Toxicology Mechanisms and Methods 21, 53-62 (2011); R. K. Sit et al., New Structural Scaffolds for Centrally Acting Oxime Reactivators of Phosphylated Cholinesterases. Journal of Biological Chemistry 286, 19422-19430 (2011)). This test product enzyme was used for evaluation using a variety of substrates to ensure that the enzyme met activity benchmarks outlined in the literature (H. Sun, Y.-P. Pang, O. Lockridge, S. Brimijoin, Re-engineering Butyrylcholinesterase as a Cocaine Hydrolase. Molecular Pharmacology 62, 220 (2002)). These assays were carried out with a standard spectrophotometer where the absorbance of 570 nm light was measured. An increase in absorbance indicates enzyme catalyzed hydrolysis of resorufin acetate. Additionally, activity of CocE was tested by monitoring the production and release of benzoic acid by monitoring the change in absorbance of 240 nm light when cocaine was incubated with CocE.

### Evaluation on CocE Activity Against Sarin

Upon confirmation that the CocE provided was stable and active against control substrates, the test product was evaluated for activity against sarin. CocE was evaluated for ability to protect AChE in a solution of dilute sarin. Sarin was diluted into buffer, and its concentration was determined by LC-MS/MS. AChE from erythrocyte membrane preparations derived from human blood was be incubated with the dilute sarin in the presence and absence of CocE. Evaluation of the timing of administration of CocE was be tested by varying the timing of addition of both sarin and CocE to the reaction. Retention of AChE activity after exposure to various concentrations of sarin and sarin with CocE was be measured using a spectrophotometric method developed by Applicant.

Several crystal structures of both BChE and CocE exist. Small molecules, such as, sarin, easily fit into the active site of each of these enzymes. In a relatively simplistic in silico modeling experiment, it was observed that hydrolysis and product release of sarin. A more rigorous set of in silico modeling experiments could shed light on either the mechanism of action or the mechanism of inhibition.

### AChE Protection Study

Human erythrocyte AChE was be incubated in assay buffer with dilute sarin in the presence and absence of the candidate MCM. The timing of addition of sarin and the candidate MCM to the reaction mixture was varied to evaluate the effects of timing of administration of the candidate MCM. Following the incubation, the AChE activity was measured using a spectrophotometric method. The incubation times were decided based on data from a similar enzymatic AChE protection assays, reported by Moyer et al. (K. G. McGarry, K. E. Schill, T. P. Winters, E. E. Lemmon, C. L. Sabourin, J. A. Harvilchuck, R. A. Moyer, Characterization of cholinesterases from multiple large animal species for medical countermeasure development against chemical warfare nerve agents. Toxicol. Sci. 2020, 174, 124-132.)

### Agent Degradation Study

The candidate MCM was incubated in a buffer with dilute sarin, and the concentration of sarin and its major metabolite(s) was measured at selected time points by LC-MS/MS. The time points were selected based on data from a similar assay, reported by Woold et al. (K. G. McGarry, R. F. Lalisse, R. A. Moyer, K. M. Johnson, A. M. Tallan, T. P. Winters, J. E. Taris, C. A. McElroy, E. E. Lemmon, H. S. Shafaat, Y. Fan, A. Deal, S. C. Marguet, J. A. Harvilchuck, C. M. Hadad, D. W. Wood, A novel, modified human butyrylcholinesterase catalytically degrades the chemical warfare nerve agent, sarin. Toxicol. Sci. 2020, 174, 133-146.)

### CocE Activity to Degrade Sariin

The ability of CocE to degrade sarin will be tested by incubation of the enzyme with dilute sarin. At various time points samples will be collected, concentration of sarin and its metabolites measured via a LC-MS/MS.

The CocE activity on the substrate sarin in both the AChE protection assay and the degradation assay can benefit from the addition of oximes. The use of oximes should not affect the ability of the LC-MS/MS assay to detect levels of sarin and its metabolites accurately. The oximes is expected to assist in the protection of AChE, but this phenomenon is to be evaluated in the context of CocE as well allowing a comparison of the effect of CocE.

### Modeling Study

Four organophosphate ligands, dichlorvos, paraoxon, naled, and sarin, were prepared for docking by generating initial conformations with corrected tautomer and ionization states. Coordinates for a 1.53 Å resolution, unliganded x-ray crystal structure of cocaine esterase (PDB ID: 3PUI) were downloaded from the Protein Data Bank. The protein was prepared for docking by performing tautomer and ionization state assignment, and residue corrections as needed. Modeling included automated and minimization docking of the molecules in two different forms of the protein, with the protons on residues in the catalytic triad active site either in their ground or activated state locations (i.e., either on serine 117 or on aspartate 259 respectively). The second step of modeling calculated the binding strain of resulting docking poses using both classical and quantum mechanics theories. Finally, the resulting docking poses were manually inspected.

Modeling software included proprietary Denovicon software in addition to the following open-source software packages: rDock (http://rdock.sourceforge.net/), MGL Tools: https://ccsb.scripps.edu/mgltools/), and Auto3D: https://github.com/isayevlab/Auto3D_pkg).

In all docking experiments, the organophosphate ligand settled into the active site containing the catalytic triad of cocaine esterase. When serine was in a protonated ground state of ionization, the phosphate of the ligand substrate was observed to be no further than 4.2 Å from the gamma oxygen on the serine side-chain. When the serine was deprotonated in an active ionization state, the phosphate was observed to be no further than 4.0 Å from the gamma oxygen of the serine side-chain. In all cases, the organophosphate was observed no further than 4.2 Å from the protonated nitrogen on histidine 287.

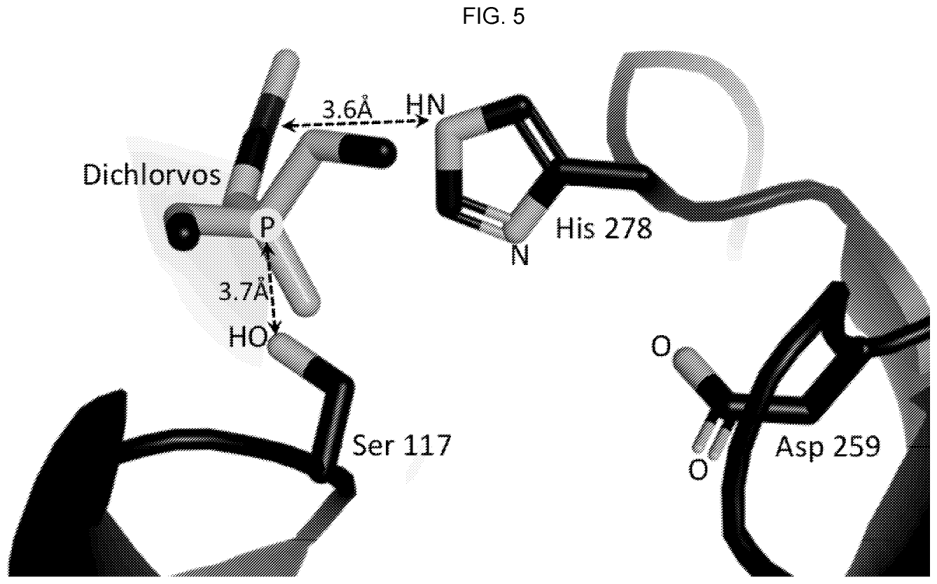

In FIG. 5, dichlorvos was docked to cocaine esterase, with phosphate group of the ligand oriented toward the catalytic triad. Intermolecular contacts were formed between the substrate phosphorous and gamma oxygen of the protonated catalytic serine (3.4 Å) as well as the substrate alkene carbons and epsilon nitrogen of histidine (3.6 Å).

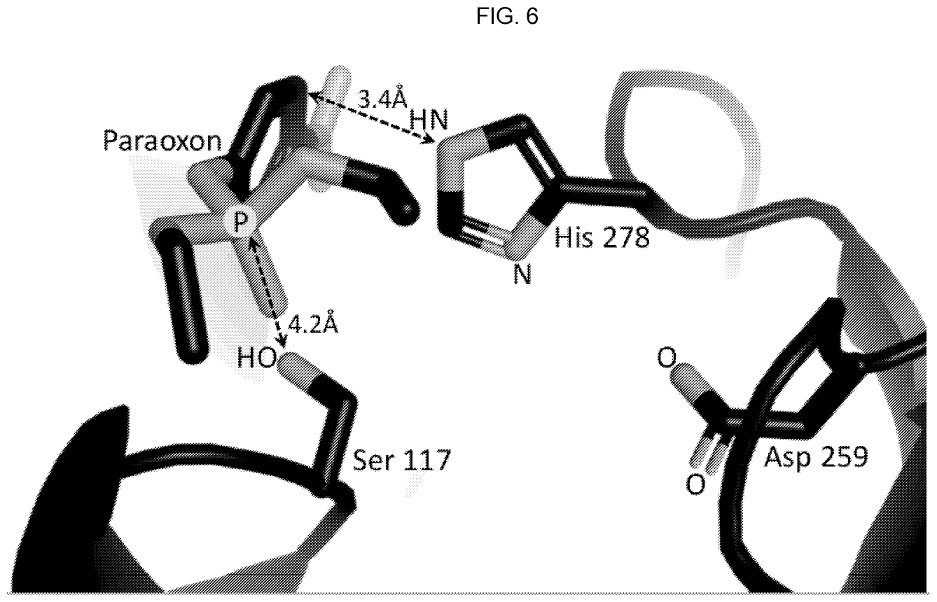

In FIG. 6, paraoxon docked to cocaine esterase, with phosphate group of the ligand oriented toward the catalytic triad. Intermolecular contacts were formed between the substrate phosphorous and gamma oxygen of the protonated catalytic serine (4.2 Å) as well as a substrate aromatic carbon and epsilon nitrogen of histidine (3.4 Å).

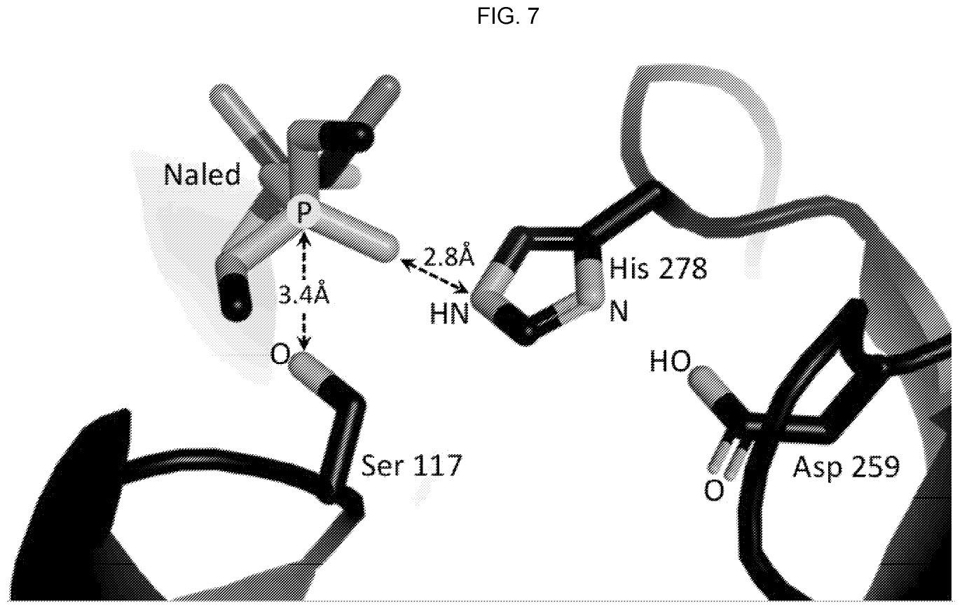

In FIG. 7, naled docked to cocaine esterase, with phosphate group of the ligand oriented toward the catalytic triad. Intermolecular contacts were formed between the substrate phosphorous and gamma oxygen of the deprotonate catalytic serine (3.4 Å), positioned for an in-line nucleophilic attack.

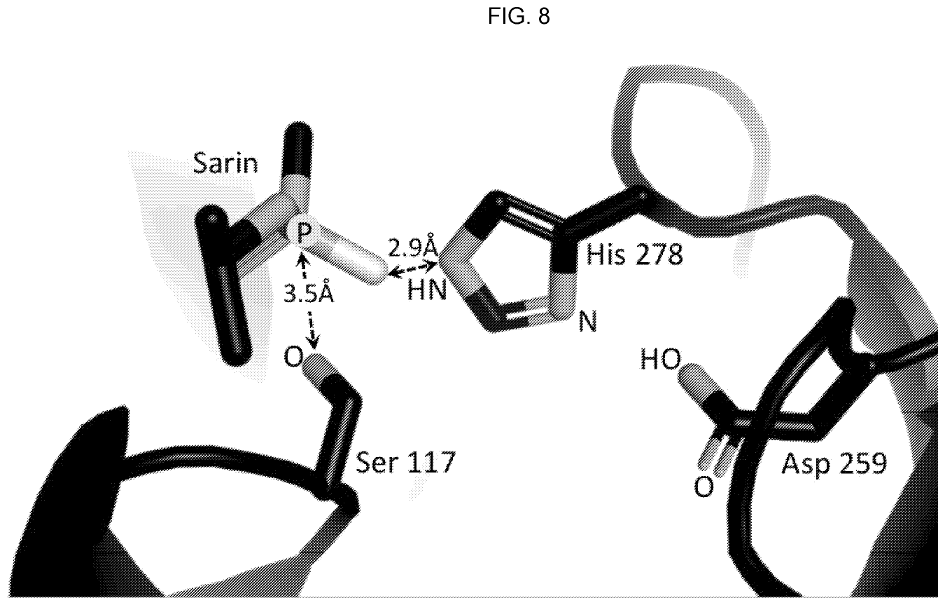

In FIG. 8, sarin docked to cocaine esterase, with phosphate group of the ligand oriented toward the catalytic triad and the propyl group nestled in a small hydrophobic cleft adjacent to the active site. Intermolecular contacts were formed between the substrate phosphorous and gamma oxygen of the deprotonated catalytic serine (3.5 Å) as well as the substrate fluorine and epsilon nitrogen of histidine (2.9 Å).

### Evaluation of Enzyme-Substrate Reaction by Mass Spectrometry

Positive control, cocaine, and experimental organophosphate substrate, dichlorvos, were separately incubated with protein at 37° C. At specific time points over a 6 hour-time course, an aliquot of the reaction was removed and quenched with 10× volume water/acetonitrile containing 0.1% formic acid to stop the reaction. The sample was briefly vortexed and then placed into a microtiter plate for analysis by LC/MS/MS. The peaks corresponding to cocaine or dichlorvos were integrated using AB SCIEX analyst 1.7 software. The integrated peak area was plotted against time to generate substrate disappearance plots.

An AB SCIEX API4000Qtrap equipped with a Shimadzu LC20AD HPLC system and CTC Analytics Leap Autosampler was used for the analysis of samples. The mass spectrometer was operated in MRM mode, allowing for selective and sensitive detection of the two analytes in positive ion mode of operation. The mass spectrometer parameters are shown in Table 2 and Table 3.

TABLE 2
Instrument Parameters for Mass Spectrometer

| Parameter | Value | |
|---|---|---|
| Scan Type | MRM | |
| Scheduled MRM | No | |
| Polarity | Positive | |
| Scan Mode | N/A | |
| Ion Source | Turbo Spray | |
| Resolution Q1 | Unit | |
| Resolution Q3 | Unit | |
| Intensity Threshold | 0.00 | cps |
| Settling Time | 0.0000 | msec |
| MR Pause | 5.0070 | msec |
| MCA | No | |
| Step Size | 0.00 | Da |
| CUR | 30.00 | |
| IS | 5000 | V |
| TEM | 450.00 | |
| GS1 | 35.00 | |
| GS2 | 35.00 | |
| ihe | ON | |
| CAD | 7.00 | |
| CAD gas | N₂ | |
| N₂ inlet pressure | 60 | psig |

| | |
|---|---|
| EP | 10.00 |

TABLE 3
MS/MS Acquisition Parameters for Mass Spectrometer

| Q1 mass | Q3 mass | Dwell | | | | |
|---|---|---|---|---|---|---|
| (Da) | (Da) | (sec) | Parameter | Start | Stop | ID |
| 221.045 | 126.800 | 50 | DP | 66.00 | 66.00 | Dichlorvos |
| | | | CE | 23.00 | 23.00 | MRM1 |
| | | | CXP | 6.00 | 6.00 | |
| 221.045 | 108.900 | 50 | DP | 66.00 | 66.00 | Dichlorvos |
| | | | CE | 25.00 | 25.00 | MRM2 |
| | | | CXP | 20.00 | 20.00 | |
| 304.200 | 182.100 | 50 | DP | 71.00 | 71.00 | Cocaine |
| | | | CE | 27.00 | 27.00 | MRM1 |
| | | | CXP | 12.00 | 12.00 | |
| 304.200 | 77.100 | 50 | DP | 71.00 | 71.00 | Cocaine |
| | | | CE | 79.00 | 79.00 | MRM2 |
| | | | CXP | 14.00 | 14.00 | |

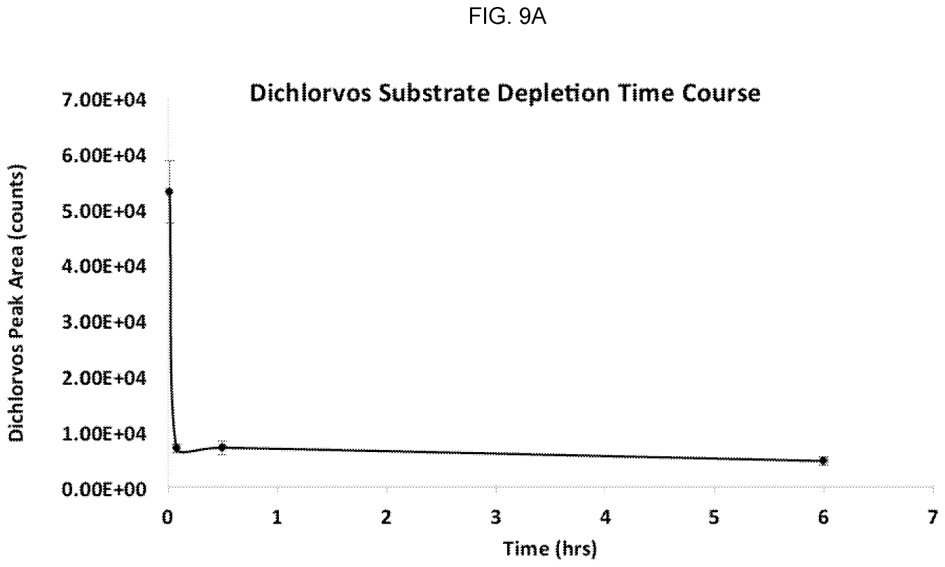

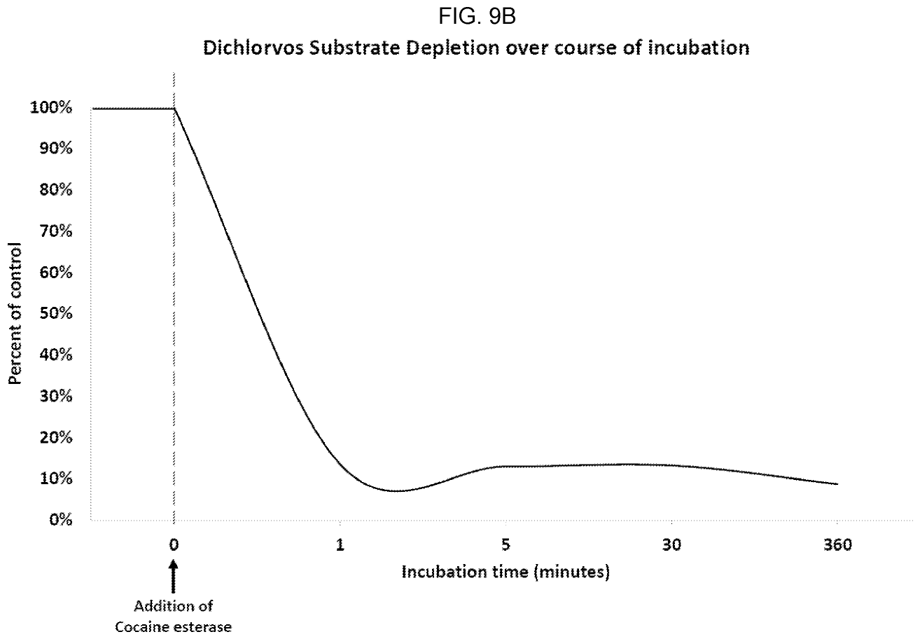

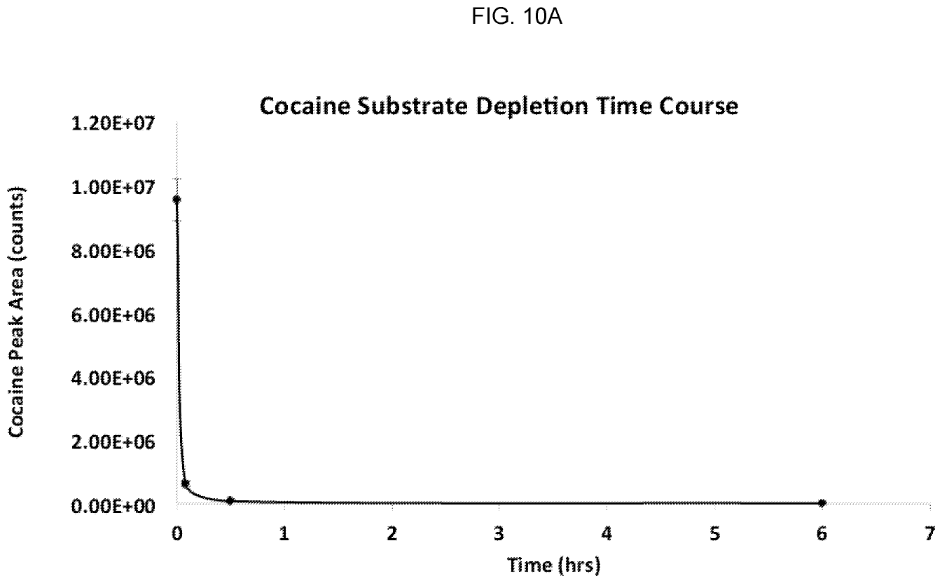

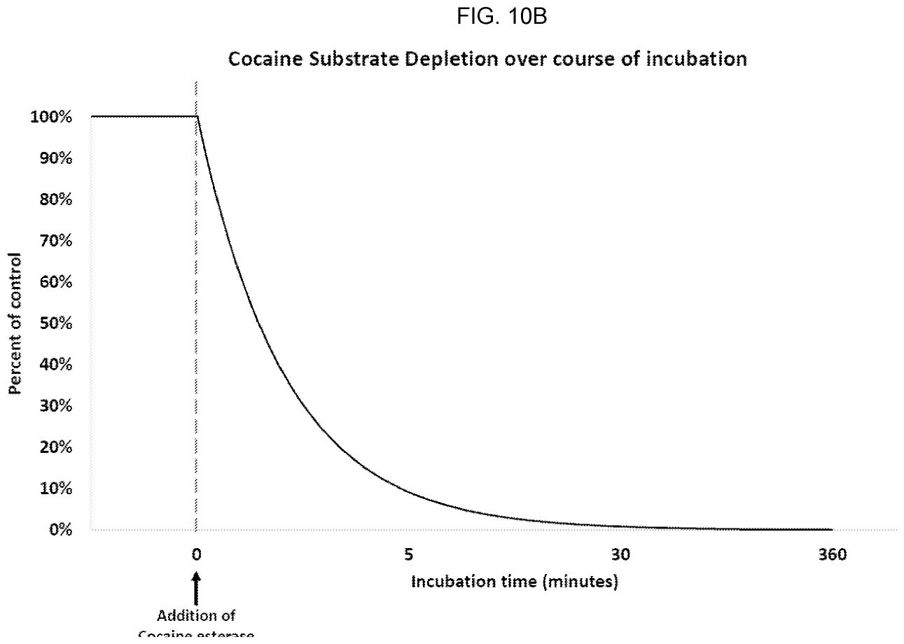

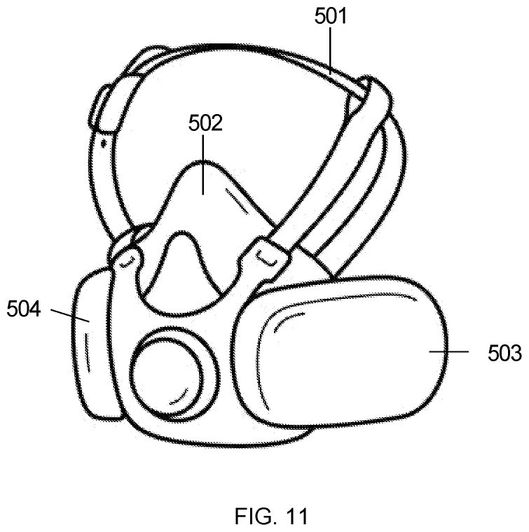

Samples of cocaine esterase and dichlorvos or cocaine were quenched at various time points over 6 hours and run on a mass spectrometry machine according to the disappearance method outlined above. Both dichlorvos and cocaine substrates were depleted to a level indistinguishable from noise within the first hour of testing. Over the remaining 5 hours, there was little observable change as the substrate had already been depleted. Dichlorvos is rapidly depleted within the first hour (FIGS. 9A and 9B). Cocaine is rapidly depleted within the first hour (FIGS. 10A and 10B).

### Regulatory Compliance

This study was conducted using good documentation practices consistent with, but not in strict accordance with, the current version of the United States Food and Drug Administration's (FDA) Good Laboratory Practice (GLP) Regulations, 21 CFR Part 58 for the conduct of non-clinical laboratory studies. All portions of this study adhered to the study protocol and any amendments, as well as to applicable Battelle facility standard operating procedures (SOPs).

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method for detoxifying an organophosphate-based agent, wherein the method comprises:
contacting the organophosphate-based agent with a cocaine esterase,
wherein the contacting detoxifies the organophosphate-based agent.

2. The method of claim 1, wherein the cocaine esterase comprises an amino acid sequence with at least two mutations in SEQ ID NO: 1 or SEQ ID NO: 3.

3. The method of claim 2, wherein the at least two mutations comprise T172R and G173Q.

4. The method of claim 2, wherein the cocaine esterase comprises a catalytic triad of aspartate, histidine, and serine in SEQ ID NO: 1 or SEQ ID NO: 3.

5. (canceled)

6. The method of claim 1, further comprising: adding an oxime compound after contacting the organophosphate-based agent with the cocaine esterase, wherein the oxime compound is selected from the group consisting of pralidoxime (2-PAM), asoxime (HI-6), deazapralidoxime (DZP), methoxime (MMB4), obidoxime, trimedoxime (TMB4), TAB2OH, ortho-7, and 3-hyroxy-2-pyridinealdoxime.

7. The method of claim 6, wherein the oxime compound assists with catalysis of a cocaine esterase-mediated hydrolysis of the organophosphate-based agent.

8. (canceled)

9. The method of claim 1, wherein the organophosphate-based agent comprises a chemical weapon, wherein the chemical weapon is selected from the group consisting of G-series nerve agents, V-series nerve agents, GV-series nerve agents, carbamates, and fourth generation agents.

10. (canceled)

11. The method of claim 1, wherein the organophosphate-based agent comprises a chemical weapon, wherein the chemical weapon is selected from the group consisting of tabun (GA), sarin (GB), butylsarin, diethyltabun, soman (GD), cyclosarin (GF), Novichok agents A232 and A234, GV, VE, VG, VM, VP, VS, venomous agent X (VX), Chinese VX, Russian VX (VR), EA-3148, EA-2192, 2-dimethylaminoethyl-(dimethylamido)-fluorophosphate), aldicarb, methomyl, EA-3990, EA-4056, substance-33, A230, Novichok-5, Novichok-7, paraoxon, paraoxon-ethyl, paraoxon-methyl, methamidophos, and fenamiphos.

12. The method of claim 1, wherein the organophosphate-based agent comprises an organophosphate-based pesticide, selected from the group consisting of azamethiphos, azinphos methyl, bomyl, carbamates (aldicarb, methomyl, EA-3990, and EA-4056), carbophenothion, chlorethoxyphos, chlorfenvinphos, chlormephos, chlorpyrifos, chlorpyrifos methyl, chlorthiophos, coumaphos, cyanofenphos, demeton, dialifor, dialkylphosphates (DAPs), diazinon, dichlorvos, dicrotophos, diethyldithiophosphate (DEDTP), diethylphosphate (DEP), dimefos, dimefox, dimethoate, dimethyldithiophosphate (DMDTP), dimethylthiophosphate (DMTP), dioxathion, disulfoton, endothion, EPN, ethion, ethyl parathion, famphur, fenamiphos, fenophosphon, fensulfothion, fenthion, fenitrothion, fonofos, fosthietan, isofenphos, 2-isopropyl-4-methyl-6-hydroxypyrimidine (IMPY), isazophos methyl, malathion, mephosfolan methamidophos, methidathion, methyl parathion, mevinphos, mipafox, monocrotophos, oxydemeton methyl, parathion (or ethyl parathion), paraoxon, phorate, phosfolan, phosmet (imidan), phosphamidon, pirimiphos methyl, prothoate, schradan, sulfotepp, temephos, terbuos, tetrachlorvinphos, tetraethyl pyrophosphate, dimethyl 1,2-dibromo-2,2-dichloroethylphosphate (naled or dibrom), and 3,5,6-trichloro-2-pyridinol (TCPy).

13. (canceled)

14. A composition comprising a therapeutically effective amount of a cocaine esterase to detoxify an organophosphate-based agent.

15. (canceled)

16. The composition of claim 14, wherein the cocaine esterase comprises an amino acid sequence with at least two mutations in SEQ ID NO: 1 or SEQ ID NO: 3.

17. The composition of claim 16, wherein the at least two mutations comprise T172R and G173Q.

18. The composition of claim 16, wherein the cocaine esterase comprises a catalytic triad of aspartate, histidine, and serine in SEQ ID NO: 1 or SEQ ID NO: 3.

19-22. (canceled)

23. The composition of claim 14, further comprising an oxime compound, wherein the oxime compound is selected from the group consisting of pralidoxime (2-PAM), asoxime (HI-6), deazapralidoxime (DZP), methoxime (MMB4), obidoxime, trimedoxime (TMB4), TAB2OH, ortho-7, and 3-hyroxy-2-pyridinealdoxime.

24-60. (canceled)

61. The method of claim 1, wherein the cocaine esterase is further PEGylated.

62. The method of claim 1, wherein the cocaine esterase is active for greater than or equal to 6 h at 37° C.

63. The method of claim 1, wherein maximum initial velocity of a reaction (Vₘₐₓ) of the cocaine esterase ranges from 1200 μmol/min to 12,000 μmol/min.

64. (canceled)

65. (canceled)

66. The method of claim 1, wherein upon contacting the cocaine esterase with the organophosphate-based agent, an amount of the organophosphate-based agent is decreased to no more than about 25% of the original amount after no more than about 30 minutes.

67. (canceled)

68. A kit for detoxifying an organophosphate-based agent on, around, or in an individual in need thereof, wherein the kit comprises:
an article or composition comprising a therapeutically effective amount of a cocaine esterase; and
instructions for wearing the article or administering a therapeutically effective amount of the composition to detoxify the organophosphate-based agent to the individual in need thereof.

69. The kit of claim 68, wherein the composition comprises cocaine esterase in a range from 50 mg to 400 mg.
